(19) United States
(12) Patent Application Publication
(10) Pub. No.: US 2010/0174218 A1
(43) Pub. Date: Jul. 8, 2010

(54) Pelvic exercise systems and methods

(76) Inventor: Youngtack Shim, Port Moody (CA)

Correspondence Address: Youngtack Shim, 155 Aspenwood Drive, Port Moody, BC, V3H 5A5, CA

(21) Appl. No.: 12/318,661

(22) Filed: Jan. 6, 2009

(51) Int. Cl. A61H 7/00; A63B 23/00; A61B 5/103

(52) U.S. Cl. 601/84; 482/148; 600/587

(57) ABSTRACT

The present invention generally relates to a pelvic exercise system for measuring at least one variable which represents physiologic state of a pelvic structure of an user and for providing a value of the variable to the user. More particularly, the present invention relates to a pelvic exercise system for measuring the pelvic variable during exercising various muscles of the pelvic structure by at least one sensor unit of the system and for providing at least one audio and/or visual feedback signal to the user, thereby allowing the user to monitor the physiologic state of her structure. Such a system may be arranged to monitor such a variable as the user maintains a preset posture of the pelvic structure or to measure variations in such a variable while the user changes the posture of the structure during exercise. The present invention also relates to various methods of measuring such a variable and for providing such a variable. The present invention also relates to various processes for making various members, units, and/or parts of the above pelvic exercise system, for measuring the variable, and for providing such a variable to the user.

### CROSS-REFERENCES TO RELATED APPLICATIONS

The present application claims an earlier invention date of the Disclosure Document entitled the same, deposited in the U.S. Patent and Trademark Office (the “Office”) on Jan. 12, 2007 under the Disclosure Document Deposit Program (“DDDP”) of the Office, and bearing the Ser. No. 611,016. The present application also claims earlier invention dates of other Disclosure Documents, the first of which is entitled “Dynamic control relaxing systems and methods,” deposited in the Office on Jan. 12, 2007 under the DDDP, and bearing the Ser. No. 611,023, the second of which is entitled “Audio relaxing systems and methods, deposited in the Office on Jan. 23, 2007 under the DDDP, and bearing the Ser. No. 611,331, and then the third of which is entitled “Synchronized relaxing systems and methods,” deposited in the Office on Jan. 12, 2007 under the DDDP, and bearing the Ser. No. 611,027. It is to be appreciated that an entire portion of each of the above Disclosure Documents is incorporated herein by reference.

### FIELD OF THE INVENTION

The present invention generally relates to a pelvic exercise system for measuring at least one variable which represents physiologic state of a pelvic structure of an user and for providing a value of the variable to the user. More particularly, the present invention relates to a pelvic exercise system for measuring the pelvic variable during exercising various muscles of the pelvic structure by at least one sensor unit of the system and for providing at least one audio and/or visual feedback signal to the user, thereby allowing the user to monitor the physiologic state of her structure. Such a system may be arranged to monitor such a variable as the user maintains a preset posture of the pelvic structure or to measure variations in such a variable while the user changes the posture of the structure during exercise. The present invention also relates to various methods of measuring such a variable and for providing such a variable. The present invention also relates to various processes for making various members, units, and/or parts of the above pelvic exercise system, for measuring the variable, and for providing such a variable to the user.

### BACKGROUND OF THE INVENTION

A vaginal anatomy typically includes a vaginal entry and a vaginal wall, where such an entry defines an orifice therethrough, while the wall includes muscles and defines a vaginal cavity which extends inwardly from the entry and which is also bound by such muscles. The entry forms a clitoris thereon, and a paraurethral gland of an urethral sponge of a clitoris (also called the Grafenberg spot or G spot) is believed to be defined on the wall. The vaginal wall is formed essentially of two sets of muscles, the former extending longitudinally while the latter encircling the vagina. These muscles are specifically termed as “pubococygenus” and “levator ani” and are located immediately adjacent to the vagina. These muscles have general appearance of a hammock with its two ends connected to the sides of a pelvis. In particular, the pubococygenus is basically a sphincter muscle which passes through a middle third of the vagina and runs in a circular band, with a ring-like ridges forming a part of a urethra and anus. For simplicity of illustration, this vaginal anatomy is to be referred to as “a pelvic structure,” the vaginal entry as an “entry,” and the vaginal wall as a “wall.” In addition, such a pelvic structure is referred to as the “standard pelvic structure” or simply the “pelvic structure” hereinafter for simplicity of illustration, unless otherwise specified.

Among devices currently available in the market for enhancing sexual functioning are dildos, vaginal exercise bars, and prostate stimulators. These devices generally provide stimuli from friction upon manipulation of their stimulators or by pressure due to distention of the pelvic cavity effected by a volume of such stimulators.

For example, manual pelvic relaxing devices have been proposed in various configurations as disclosed in various prior art such as, e.g., U.S. Pat. No. 3,996,930 to Sekulich, U.S. Pat. No. 5,690,603 to Kain, U.S. Pat. No. 5,690,604 to Barnett, U.S. Pat. No. 5,853,362 to Jacobs, U.S. Pat. No. 6,203,491 to Uribe, U.S. Pat. App. Pub. No. 2005/0187431 by Hudson, U.S. Pat. App. Pub. No. 2005/0228218 by Skidmore et al., U.S. Pat. No. 6,540,667 to Hickman, and the like. Being manual, users have to manually move such devices in and out of the pelvic cavity and/or around the pelvic opening.

In order to overcome inconvenience thereof, various automatic mechanisms have been added to the pelvic relaxation devices. In one class of examples, electric motors or electromagnetic vibration mechanisms have been incorporated to various automatic devices for effecting vibration as disclosed in various prior art such as, e.g., U.S. Pat. No. 3,451,391 to Tavel, U.S. Pat. No. 3,504,665 to Bakunin et al., U.S. Pat. No. 3,626,931 to Bysakh, U.S. Pat. No. 3,669,100 to Csanad, U.S. Pat. No. 3,991,751 to O'Rourke, U.S. Pat. No. 4,788,968 to Rudashevsky et al., U.S. Pat. No. 5,067,480 to Woog et al, U.S. Pat. No. 6,056,705 to Stigar-Brown, U.S. Pat. Appl. Pub. 2004/0034315 to Chen, U.S. Pat. Appl. Pub. 2004/0127766 to Chen, and the like.

In another class of example, automatic pelvic relaxing devices have used various mechanisms of converting rotational movements generated by such electric motors into translational movements for effecting horizontal and/or vertical translation of their stimulators. Several examples of such prior art include U.S. Pat. No. 4,722,327 to Harvey, U.S. Pat. No. 4,790,296 to Segal, U.S. Pat. No. 5,076,261 to Black, U.S. Pat. No. 5,725,473 to Taylor, U.S. Pat. No. 6,142,929 to Padgett, U.S. Pat. No. 6,422,993 to Hudson, U.S. Pat. No. 6,866,645 to Lee, and U.S. Pat. Appl. Pub. 2004/0147858

Various automatic pelvic relaxing devices have also used various mechanisms for converting rotational movements generated by such electric motors into lateral movements for effecting horizontal translation of their stimulators along a direction generally normal to axes of such stimulators. Several examples of such prior art are U.S. Pat. No. 5,460,597 to Hopper, U.S. Pat. No. 5,470,303 to Leonard et al., and U.S. Pat. No. 5,851,175 to Nickell.

Other therapeutic devices, although developed for various purposes other than pelvic relaxing, seem to have been used as alternatives as such conventional pelvic relaxing devices. In one class of examples, various manual or vibration devices have been disclosed to train or heal pelvic muscles as exemplified in U.S. Pat. No. 3,598,106 to Buning, U.S. Pat. No. 4,241,912 to Mercer et al., and U.S. Pat. No. No. 4,574,791 to Mitchener. In another class of examples, various devices have been developed for massaging various portions of a human body as disclosed in U.S. Pat. No. 4,055,170 to Nohmura, U.S. Pat. No. 4,825,853 to Iwamoto et al., U.S. Pat. No. 4,846,158 to Teranishi, U.S. Pat. No. 4,911,149 to Borodulin et al., U.S. Pat. No. 5,063,911 to Teranishi, and the like. In another class, massage devices have also been devised to provide translational movements as disclosed in U.S. Pat. No. 4,002,164 to Bradley, U.S. Pat. No. 5,085,207 to Fiore, U.S. Pat. No. 5,676,637 to Lee, and the like. In another class of examples, various devices have also been arranged to provide rotating, tapping, swinging and/or swiveling movements as described in U.S. Pat. No. 4,162,675 to Kawada, U.S. Pat. No. 6,632,185 to Chen, U.S. Pat. No. 4,088,128 to Mabuchi, U.S. Pat. No. 4,513,737 to Mabuchi, U.S. Pat. No. 4,827,914 to Kamazawa, U.S. Pat. No. 4,834,075 to Guo et al., U.S. Pat. No. 5,183,034 to Yamasaki et al., and U.S. Pat. No. 6,402,710 to Hsu. A vacuum device of U.S. Pat. No. 4,033,338 to Igwebike as well as a balloon device of U.S. Pat. No. 4,050,449 to Castellana et al. have also been proposed.

Regardless of their detailed mechanisms and/or movements effected thereby, all of these prior art devices suffer from common drawbacks. Excluding those manual ones, typical automatic devices consist of main modules and control modules which operatively couple with the main modules by wire for delivering electric power and control signals. Such wire, however, tends to be easily tangled and damaged. To overcome this defect, modern automatic pelvic relaxing device are fabricated as single unitary articles each with a main body and a handle which fixedly couples with a top part of the main body. The main body is generally designed to be inserted into the pelvic cavity, whereas the handle is shaped and sized to provide a grip for the user and also incorporates therein various control buttons. Accordingly, the handle consists of a space to form the grip and another space for such buttons. In order to avoid providing an inadvertently long device, however, a part of the handle closer to the main body is recruited to define the grip, whereas the rest of the handle houses the control buttons. It is to be appreciated, however, that all control buttons of conventional automatic devices are either on/off switches or speed control switches, where the on/off switches turn on and off the entire device or a specific movement thereof, and the speed control switches control a speed of the specific movement. In addition, such switches are typically designed to be activated and deactivated each time the user presses or touches them. Accordingly, when the user inadvertently touches any of such on/off and control switches during use, the device may be accidentally turned off, change speeds, and the like. In order to avoid such inadvertent operation, the control buttons have been incorporated as far away from the grip space of the handle, which in turn causes the very inconvenience of requiring the user to change the grip or to move his or her hand to manipulate the control buttons during operation when the user wants to change the speed of movement.

In contrary to these devices, novel pelvic relaxing systems, methods, and/or processes have already been conceived of and disclosed in numerous co-pending applications of the same Applicant. For example, various pelvic relaxing systems have been proposed for manipulating their input and/or sensor units without mandating the user to change the grip, for providing various stimuli to the clitoris and/or G-spot of the user, for providing interactive capabilities thereto, for synchronizing movements of their various parts and/or operations thereof with internal and/or external signals, for incorporating electric stimulators thereinto, for installing the body members capable of adjusting their configurations, for incorporating retention mechanisms thereinto, for providing feedback mechanisms thereto, and for generating reciprocating movements of only portions of body members thereof. Although these novel systems solve most deficiencies of the conventional devices, none of them are capable of assisting the user in improving muscle tones of her pelvic structure.

It is well recognized in the field that improved muscle strengths and tones of various muscles of the pelvic structure are advantageous in many respects. For example, the stronger pelvic muscles are generally attributed to experiencing an orgasm or to having more intense and/or multiple orgasms during a sexual intercourse. To this end, various modalities have been proposed to improve tones of the pelvic muscles among which the most popular is the “Kegel” exercise. Similar to other exercising modalities, the Kegel exercise hinges on a well-accepted principle of “Specific Adaptation to Imposed Demand” which dictates that muscles will adapt only to demands placed thereupon. Accordingly, one need to place an ever-increasing demand on her muscles for continued improvement thereof, e.g., by adding an extra repetition to an exercise, increasing demand (or load) on the muscles, shortening rest intervals during the exercise, and the like. Accordingly, various conventional pelvic exercise devices urge the user to add progressive dynamic resistance to conventional Kegel exercise devices.

Although conventional Kegel exercise devices are generally effective in improving the muscle strengths and tones, such devices suffer from several common deficiencies. For example, almost all conventional devices use sensors filled with air, and assess physiologic states of various muscles by monitoring changes in pressure of air trapped inside the sensors. The sensor is generally disposed in an insertable unit of the device and arranged to be in fluid communication with a monitoring unit such as an analog pressure gauge or a digital display through a tubing which is typically made of polymers. Because air is a compressible gas, however, such air pressure inside the sensor generally depends upon temperature of a surrounding medium. Therefore, the air trapped in the sensor is heated to body temperature upon insertion of the insertable unit into the internal cavity of the structure, and expands due to an increase in temperature. Because the tubing is disposed in room temperature, however, a mismatch between such temperatures may cause measurement error in the air pressure. In addition, the compliant tubing between the sensor and monitoring unit may change its internal volume when the tubing is moved around. In addition, such a conventional device is typically forced to employ a single sensor, for it is not readily amenable to incorporate two compliant air-filled sensors. Therefore, such a device employs the sensor over at least a substantial part of its insertable unit and only provides a value of the air pressure averaged over the part of the sensor. Moreover, such conventional devices are directed to measure the pressure exerted by the pelvic muscles while the user maintains a fixed posture and cannot assess configurational changes in the pelvic structure as the user is in a different posture.

Accordingly, there is a need for a pelvic exercise system capable of measuring various pelvic variables more accurately and/or dynamically. In addition, there is a need for a pelvic exercise system capable of assessing temporal and/or spatial distribution of such variables inside the pelvic structure. There also is a need for a pelvic exercise system capable of allowing the user to assess changes in various pelvic variables based on various postures of the pelvic structure. Moreover, there is a need for a pelvic exercise system capable of moving into or out of such a pelvic structure and facilitating an user to exercise against such movement.

### SUMMARY OF THE INVENTION

The present invention generally relates to a pelvic exercise system for measuring at least one variable which represents physiologic state of a pelvic structure of an user and for providing a value of the variable to the user. More particularly, the present invention relates to a pelvic exercise system for measuring the pelvic variable during exercising various muscles of the pelvic structure by at least one sensor unit of the system and for providing at least one audio and/or visual feedback signal to the user, thereby allowing the user to monitor the physiologic state of her structure. Such a system may employ various sensor units each of which may be capable of monitoring force, velocity, acceleration, displacement, duration, frequency, and electric voltages and/or current related to the pelvic muscles, where the variables may be effected by the pelvic structure or where such variables may represent reaction or resistance to various movements of the system. The system may also generate various feedback audible and/or visual signals so that the user may audibly monitor the value of the variables or that the user may perform exercise while synchronizing contraction and relaxation of the structure with such feedback signals. Such a system may be arranged to monitor the variable while the user maintains a preset posture of the pelvic structure or to measure variations in such a variable while the user changes the posture of the structure during exercise.

The present invention relates to various methods of measuring various variables representing the state of the pelvic structure and of providing the user with values of such pelvic variables. More particularly, the present invention relates to various methods for measuring various pelvic variables, various methods for acquiring values of the variables in a single portion and/or multiple portions of the structure, for generating audible and/or visual feedback signals during exercising the pelvic structure, for synchronizing such exercising with the audible and/or visual signals representing such variables, for measuring multiple variables in a single portion or multiple portions of the structure using a single or multiple sensor units, for fitting the sensor unit onto the structure during measuring the variables, and the like. The present invention also relates to various methods of improving strengths and/or tones of the pelvic muscles, of assessing such variables in a preset posture of the structure, of assessing a change in such variables as the user changes the posture, of identifying the posture and/or a series of such postures in which one or more pelvic variables may define desirable values, and the like. The present invention also relates to various processes for making various members, units, and/or parts of such pelvic exercise systems, for measuring various variables by such sensor units, for providing the values of such variables in various modes, and the like.

Therefore, one objective of the present invention is to provide a pelvic exercise system which may allow an user to improve strengths and/or tones of her pelvic muscles such as, e.g., pelvic floor muscles and others. Thus, a related objective of the present invention is to provide the system which may include one or more sensor units for measuring various pelvic variables representing physiologic states of such a structure to the user and providing values of the variables thereto during exercising such muscles.

Another objective of the present invention is to provide another pelvic exercise system which may measure various variables such as normal force applied onto at least a part of the system or onto the portion of the structure, bending force applied to such a part or such a portion, axial force pulling (or pushing) the part into (or out of) the internal cavity of the structure, torque applied around the part or portion, velocity of the part or portion, acceleration of the part or portion, displacement of the part or portion, contact between such a part and portion, a dimension of the portion, contraction or relaxation of the portion, a duration and/or a frequency of at least one of such variables, and the like.

Another objective of the present invention is to provide another pelvic exercise system which may allow the user to exercise her pelvic muscles against load units capable of offering various loads of resistance, viscosity, and/or mass. Thus, a related objective of this invention is to provide the load unit which may adjust such resistance, viscosity, and/or mass. Another related objective is to provide the load unit which may be replaced or added by another load unit and which may therefore allow the user to manipulate such resistance, viscosity, and/or mass.

Another objective of the present invention is to provide another pelvic exercise system which may incorporate a single sensor unit or multiple sensor units each measuring one or multiple variables in different portions of the pelvic structure. Thus, a related objective of this invention is to provide the system which may include a single sensor unit for measuring such a variable over time and assessing temporal pattern thereof, for measuring such a variable across different portions of the structure and assessing spatial distribution thereof, and the like.

Another objective of the present invention is to provide another pelvic exercise system which may include multiple sensor units and measure a similar variable or different variables across multiple portions of the structure. Thus, a related objective of this invention is to provide the system capable of measuring the variable(s) which may be effected onto (or by) different portions of the system and providing spatial distribution of such variable(s) to the user in the structure.

Another objective of the present invention is to provide another pelvic exercise system which may provide the user with various feedback signals which may represent the physiologic state of the structure. Thus, a related objective of this invention is to provide the system capable of generating an audible signal and/or a visual signal such as, e.g., sounds and/or images, representing such variables, accompanying content signals reflecting such variable or content signals instructing the user, and the like. Conversely, a related objective of this invention is to provide a system capable of generating the audible or visual signals which may aid the user to exercise her pelvic structure while synchronizing her exercise with such signals.

Another objective of the present invention is to provide another pelvic exercise system which may provide the user with the foregoing feedback signals with reference to an anatomy of the pelvic structure. Thus, a related objective of this invention is to provide the system capable of displaying the feedback signals which may be superposed onto a stationary or fixed image of the structure, thereby providing the user with such signals referenced or compared with various portions of the structure. Another related objective is to provide the system capable of monitoring configurational changes in the structure and displaying the feedback signals onto a time-varying or posture-specific image of such a structure, thereby providing the user with such signals spatially synchronized with various portions of the structure.

Another objective of the present invention is to provide another pelvic exercise system which may be fitted onto the pelvic wall after being inserted into the pelvic cavity and measure the variables while contacting or touching the portion of the structure. Thus, a related objective of this invention is to provide the system capable of deforming (e.g., expanding or shrinking) at least one sensor unit for contacting or touching such a portion. Another related objective is to provide the system capable of moving (e.g., extending or retracting) at least a part thereof for contacting or touching such a portion.

Another objective of the present invention is to provide another pelvic exercise system which may be fitted onto the pelvic wall after being inserted into the pelvic cavity and transmit the variables to the sensor unit which may be disposed in a part not fitted onto the pelvic wall. Therefore, a related objective of this invention is to provide the system with at least one movable part vertically or helically extending toward and retracting away from the pelvic wall by manual manipulation thereof. Another related objective is to provide the system with the movable part which may be extended automatically toward the pelvic wall until such a part contacts the wall, until such a part is abutted by the wall by a preset force, and the like.

Another objective of the present invention is to provide another pelvic exercise system which may measure an extent of bending thereof and provide the user with such an extent. Thus, a related objective of this invention is to provide the system with at least one flexible part which may be bent in a vertical or axial direction in response to the bending force (or pressure) and/or misaligned force (or pressure), and assess such an extent therefrom. Another related objective is to provide the system which may be at least substantially rigid but have the sensor unit capable of measuring the bending or misaligned force (or pressure) for assessing such an extent.

Another objective of the present invention is to provide another pelvic exercise system with at least one sensor unit which may be made of and/or include at least one variable resistance article and measure the variable based on electrical resistance of such an article. Therefore, a related objective of this invention is to fabricate the sensor unit including at least one movable part so that the electrical resistance of the sensor unit may be determined by a location of the movable part and that the system may measure the variable by a value of such resistance.

Another objective of the present invention is to provide another pelvic exercise system with at least one sensor unit which may be made of and/or include at least one conductive foam and measure the variable based on electrical resistance of such a foam. Thus, a related objective of this invention is to provide the sensor unit of such a conductive foam so that the electrical resistance of the sensor unit may be determined by a dimension of the foam and that the system may measure the variable by a value of such resistance. Another related objective is to provide the system with multiple sensor units of such conductive foams such that the system may measure multiple different variables thereby, that the system may measure the same variable or similar variables in different portions of the structure, and the like. Therefore, a yet another related objective is to provide the system having multiple sensor units of the conductive foams such that the system may assess spatial distribution of one or multiple variables along or across the structure.

Another objective of the present invention is to provide another pelvic exercise system which may move into (or inwardly) or out of (or outwardly) the pelvic cavity and measure variables resisting movement of the system. Therefore, a related objective of this invention is to provide the system in a preset configuration so that, once at least a part of the system is inserted into the cavity, the system may move into or out of the cavity due to the specific configuration thereof, while measuring various variables resisting the movement of thereof. Another related objective is to provide the system with an actuator member capable of moving the system into or out of the cavity so that, once inserted into the cavity, the actuator member may generate movement of the system thereinto or out thereof, while measuring the variables resisting the movement of the system.

Another objective of the present invention is to provide another pelvic exercise system which may measure various variables with one or more sensor units while the user contracts such muscles of the pelvic structure and maintains a fixed posture. Therefore, a related objective of this invention is to provide the system capable of measuring such variables in a preset posture without contracting other muscles of the user.

Another objective of the present invention is to provide another pelvic exercise system which may measure the variables with one or more sensor units while the user contracts the pelvic muscles and changes postures. Thus, a related objective of this invention is to provide the system capable of measuring the same variable(s) while the user changes the postures. Another related objective is to provide the system capable of monitoring changes in configurations the pelvic structure such as, e.g., a length, a diameter, and/or a curvature as the user changes the postures.

Another objective of the present invention is to provide another pelvic exercise system which may measure the variables by one or more sensor units while the user contracts and/or relaxes other muscles such as, e.g., abdominal muscles, muscles of her legs and/or thighs, and the like. Therefore, a related objective of this invention is to provide the system for measuring the variables while the user changes the postures and contracts various pelvic and non-pelvic muscles.

Various aspects and/or embodiments of various systems, methods, and/or processes of this invention will now be described, where such aspects and/or embodiments only represent different forms. Such systems, methods, and/or processes of this invention, however, may also be embodied in many other different forms and, therefore, should not be limited to the aspects and/or embodiments which are set forth herein. Rather, various exemplary aspects and/or embodiments described herein are provided so that this disclosure will be thorough and complete, and fully convey the scope of the present invention to one of ordinary skill in the art. It is to be understood that various movements and mechanisms therefor as well as various control algorithms of the prior art devices as described in the above Background of the Invention are to be incorporated herein in their entireties by reference.

In one aspect of the present invention, a pelvic exercise system may be arranged to measure at least one variable which may reflect a physiologic state of at least a portion of an exercising pelvic structure of an user and to provide a value of the variable to the user, where the pelvic structure may form an entry and a wall, where such an entry may be arranged to define an orifice therethrough, and where the wall may be arranged to include muscles and to form an internal cavity extending inwardly and bound by such muscles. It is to be understood that such a pelvic structure is to be referred to as a “standard pelvic structure” or a “standard structure” hereinafter for simplicity of illustration.

In one exemplary embodiment of this aspect of the invention, a system may include at least one body member and at least one control member. The body member may include a first unit which may be capable of contacting the portion of the structure when engaged therewith. Such a body member is to be referred to as a “standard body member” hereinafter for simplicity of illustration. The control member may include a single sensor unit and at least one control unit, where the sensor unit may then measure the variable, while the control unit may be operatively coupled to the sensor unit and provide the value of the variable to the user. In one example, such a variable may be normal force applied to at least a part of the first unit, bending force applied to the part, axial force pulling (or pushing) the part into (or out of) the internal cavity, torque applied around the part, velocity of the part, acceleration of the part, displacement of the part, contact between the part and portion of the structure, a dimension of such a portion, contraction and relaxation of the portion, a duration of at least one of the variables, a frequency of at least one of the variables, and the like. In another example, such a variable may be normal force applied by the portion of the structure, bending force applied by the portion, axial force generated by the structure and resisting movement of the first unit into (or out of) the cavity thereof, torque generated by the portion, velocity of such a portion, acceleration of the portion, displacement of the portion, contact between the portion and part of the of the first unit, contraction and relaxation of the portion, a duration of at least one of the variables, a frequency of at least one of the variables, and the like.

In another exemplary embodiment of this aspect of the invention, a system may include at least one standard body member and at least one control member with at least one sensor unit and at least one control unit. Such a sensor unit may measure the variable, while the control unit may operatively couple with the sensor unit and provide such a value of the variable to the user. In one example, the variable may be normal force applied to at least a part of the first unit, bending force applied onto the part, axial force pulling (or pushing) the part into (or out of) the internal cavity, torque applied around the part, velocity of the part, acceleration of the part, displacement of the part, contact between the part and portion of the structure, a dimension of the portion, contraction and relaxation of the portion, a duration of at least one of the variables, a frequency of at least one of the variables, and the like. In another example, the variable may be normal force applied by such a portion of the structure, bending force applied by the portion, axial force generated by the structure and resisting movement of the first unit into (or out of) the cavity of the structure, torque generated by the portion, velocity of the portion, acceleration of the portion, displacement of the portion, contact between such a portion and the part of the first unit, contraction and relaxation of the portion, a duration of at least one of the variables, a frequency of at least one of the variables, and the like. For each of these examples, the sensor unit may vary its configuration (or deform) while measuring the variable or, in the alternative, may at least substantially maintain its configuration while measuring the variable. For each of these example, the sensor unit may move while measuring such variable, may move along with the sensor unit while measuring the variable with the sensor unit, and the like. For each of these examples, the control unit may assess at least one another variable from the measured variable.

In another exemplary embodiment of this aspect of the invention, a system may include at least one standard body member and at least one control member with at least one sensor unit and at least one control unit. In one example, the sensor unit may monitor force (or pressure) applied thereto, and the control unit may be operatively coupled to the sensor unit and provide the value of such force (or pressure) to the user. In another example, the sensor unit may measure force (or pressure) applied to the portion of the pelvic structure, and the control unit may operatively couple with the sensor unit and provide the value of the force (or pressure) to the user. For each of such examples, the sensor unit may change its configuration (or deform) while monitoring the force (or pressure) or, alternatively, may at least substantially maintain its configuration while measuring the force (or pressure). For each of these examples, the sensor unit may move while measuring the displacement or, in the alternative, at least a part of the first unit may move along with the sensor unit while the sensor unit measures the force (or pressure). In each of such examples, the control unit may also assess acceleration of such a sensor unit, its velocity, its displacement, its mass, its momentum, its mechanical energy, a duration of the force (or pressure), and/or a frequency of the force (or pressure) from the force (or pressure) applied to the sensor unit. Alternatively, the control unit may assess acceleration of such a portion, its velocity, its displacement, its mass, its momentum, its mechanical energy, a duration of movement of the portion, and/or a frequency of the movement from the force (or pressure) applied to the portion of the structure.

In another exemplary embodiment of this aspect of the invention, a system may include at least one standard body member and at least one control member with at least one sensor unit and at least one control unit. In one example, the sensor unit may measure displacement thereof, and the control unit may operatively couple with the sensor unit and provide the value of the displacement to the user. In another example, the sensor unit may measure displacement of the portion of the structure, and the control unit may operatively couple with the sensor unit and provide the value of the displacement to the user. For both of these examples, the sensor unit may vary its configuration (or deform) while measuring the displacement or, alternatively, may at least substantially maintain its configuration while measuring the displacement. For both of such examples, the sensor unit may move while measuring the displacement or, alternatively, at least a part of the first unit may move along with the sensor unit while the sensor unit measures such displacement. For each of such examples, the control unit may assess acceleration of the sensor unit, its velocity, its mass, force (or pressure) applied onto such a sensor unit, its momentum, its mechanical energy, a duration of movement of the sensor unit effecting the displacement, and/or a frequency of the movement from the displacement of the sensor unit. For each of these examples, the control unit may assess acceleration of the portion, its velocity, its mass, force (or pressure) exerted by the portion of the structure, its momentum, its mechanical energy, a duration of movement of the portion effecting the displacement, and/or a frequency of the movement from the displacement of the portion of the structure.

In another exemplary embodiment of this aspect of the invention, a system may include at least one standard body member and at least one control member. In one example, such a sensor unit may measure velocity thereof, and the control unit may operatively couple with the sensor unit and provide the value of the velocity to the user. In another example, the sensor unit may measure velocity of the portion of the structure, and the control unit may similarly operatively couple with the sensor unit and provide the value of the velocity to the user. In each of such examples, the sensor unit may vary its configuration (or deform) while measuring the velocity or, in the alternative, may at least substantially maintain its configuration while measuring the velocity. In each of such examples, the sensor unit may move while measuring the velocity or, alternatively, at least a part of the first unit may move along with the sensor unit while the sensor unit measures the velocity. For both examples, the sensor unit may measure a distance to the structure therefrom and measure the velocity from a temporal change in the distance. For each of these examples, the control unit may assess acceleration of the sensor unit, its displacement, its mass, force (or pressure) applied onto the sensor unit, its momentum, its mechanical energy, a duration of movement of the sensor unit which causes the velocity, and/or a frequency of the movement from the velocity of the sensor unit. For each of these examples, the control unit may assess acceleration of such a portion of the structure, displacement of the portion, its mass, force (or pressure) generated thereby and applied onto the sensor unit, its momentum, its mechanical energy, a duration of movement of the sensor unit effecting the velocity, and/or a frequency of such movement from the velocity of the portion.

In another exemplary embodiment of this aspect of the invention, a system may include at least one standard body member and at least one control member. In one example, such a sensor unit may measure acceleration thereof, and the control unit may be operatively coupled to the sensor unit and provide the value of the acceleration to the user. In another example, such a sensor unit may monitor acceleration of such a portion of the structure, while the control unit may operatively couple with the sensor unit and provide the value of the acceleration to the user. For both examples, the sensor unit may change its configuration (or deform) while measuring the acceleration or, in the alternative, may at least substantially maintain its configuration while measuring the acceleration. For each example, the sensor unit may move while measuring the acceleration or, alternatively, at least a part of the first unit may move along with the sensor unit while the sensor unit measures the acceleration. For each of these examples, the sensor unit may also measure a distance to the structure therefrom, assess velocity from a temporal change in the distance, and assess the acceleration from a temporal change in the velocity. In each of such examples, the control unit may assess velocity of the sensor unit, its displacement, its mass, force (or pressure) applied onto the sensor unit, its momentum, its mechanical energy, a duration of movement of the sensor unit effecting such acceleration, and/or a frequency of the movement. For each example, the control unit may assess velocity of the portion of the structure, its displacement, its mass, force (or pressure) generated thereby and applied to such a portion of the structure, its momentum, its mechanical energy, a duration of movement of the portion effecting such acceleration, and/or a frequency of the movement from the acceleration of the portion.

In another exemplary embodiment of this aspect of the invention, a system may include at least one standard body member and at least one control member including at least one sensor unit and at least one control unit. The sensor unit may measure at least one variable representing its contact with the structure, while the control unit may operatively coupled to the sensor unit and provide a presence and/or absence of such contact to the user. In one example, the sensor unit may assess the contact through a mechanical contact between the sensor unit and such a portion of the structure. In another example, the sensor unit may assess the contact by a change in electric property around the sensor unit. In another example, the sensor unit may also assess the contact by a change in optical property around the sensor unit. In yet another example, the control unit may assess a duration of the contact and/or a frequency thereof from the presence and absence of the contact.

In another exemplary embodiment of this aspect of the invention, a system may include at least one standard body member and at least one control member including at least one sensor unit and at least one control unit. The sensor unit may monitor a duration of the variable applied onto (or effected by) the sensor unit, while the control unit may operatively couple with the sensor unit and provide the duration to the user.

In another exemplary embodiment of this aspect of the invention, a system may include at least one standard body member and at least one control member including at least one sensor unit and at least one control unit. The sensor unit may measure electric voltage and/or current generated by the muscles during their exercise and provide the at least one of the voltage and current to the user.

In another aspect of the present invention, a pelvic exercise system may measure at least one variable reflecting a physiologic state of at least a portion of the exercising standard pelvic structure of an user by at least one sensor unit incorporated into various locations thereof.

In one exemplary embodiment of this aspect of the invention, a system may include at least one standard body member and at least one control member including at least one sensor unit and at least one control unit. In one example, the sensor unit may measure the variable over at least a substantial area, a preset area, and/or a preset part of the first unit. In another example, the sensor unit may be disposed along a circumference or along a longitudinal axis of the first unit and measure the variable. In another example, the sensor unit may be helically disposed around the first unit and measure such a variable. For each of these examples, the control unit may operatively couple with the sensor unit and provide the value of the variable to the user.

In another exemplary embodiment of this aspect of the invention, a system may include at least one standard body member and at least one control member having multiple sensor units and at least one control unit. Each of the sensor units may be one of a first sensor unit which may measure such a variable over at least a substantial area of the first unit, a second sensor unit which may measure the variable over a preset area and/or part of the first unit, a third sensor unit which may be disposed along a circumference or along a longitudinal axis of the first unit and measure such a variable, and a fourth sensor unit which may be helically disposed around the first unit and measure such a variable. The control unit may be operatively coupled to each of the sensor units and provide the value of the variable to the user.

In another exemplary embodiment of this aspect of the invention, a system may include at least one standard body member and at least one control member having multiple sensor units and at least one control unit. In one example, the sensor units may measure the same variable in different parts of the first unit, while the control unit may operatively couple with the sensor units and provide the user with values of the variable measured in the different parts to the user. In another example, the sensor units may measure different variables in a preset part of such a first unit, while the control unit may be operatively coupled to such sensor units and provide values of the variables in the part to the user. In another example, such sensor units may measure different variables in different parts of the first unit and the control unit may operatively couple with the sensor units and provide values of such variables to the user.

In another aspect of the present invention, a pelvic exercise system may measure at least one variable reflecting a physiologic state of at least a portion of the exercising standard pelvic structure of an user while contacting therewith and providing a value of the variable to the user.

In one exemplary embodiment of this aspect of the invention, a system may include at least one standard body member and at least one control member. In one example, such a control member may include at least one sensor unit and at least one control unit, where such a sensor unit may be inflated toward and deflated away from the structure and monitor such a variable while contacting the portion of the structure, while the control unit may operatively couple with the sensor unit and provide such a value of the variable to the user. In another example, the control member may include multiple sensor units and at least one control unit, where each of such sensor units may monitor the same variable or different variables, where at least one of the sensor units may be inflated toward and deflated away from the structure and measure the variable while contacting the portion of the structure, and where the control unit may operatively couple with the sensor units and provide the value of the variable to the user.

In another exemplary embodiment of this aspect of the invention, a system may include at least one body member and at least one control member. In one example, the body member may include at least one first unit which may be inflated toward and/or deflated away from the structure and contact the portion of the structure when engaged therewith. In another example, the body member may have at least one first unit which may move toward and away from the structure and contact the portion of the structure when engaged therewith. For both examples, the control member may have at least one sensor unit and at least one control unit, where the sensor unit may be coupled to the first unit, move toward and away from the structure along with the first unit, and monitor the variable, and where the control unit may be operatively coupled to the sensor unit and provide the value of the variable to the user. In the alternative and for each of the examples, the control member may include multiple sensor units and at least one control unit, where each sensor unit may measure one of the same variable and different variables, where at least one of the sensor units may couple with the first unit, move toward and away from the structure along with the first unit, and monitor the variable, and where the control unit may operatively couple with the sensor unit and provide the value of the variable to the user.

In another exemplary embodiment of this aspect of the invention, a system may include at least one body member and at least one control member. In one example, the body member may include at least one first unit which may be inflated toward and/or deflated away from such a structure and may contact the portion of the structure when engaged therewith and transmit the variable therethrough. In another example, the body member may include at least one first unit which may move toward and away from the structure and may also contact the portion of the structure when engaged therewith and send the variable therethrough. For both examples, the control member may include at least one sensor unit and at least one control unit, where the sensor unit may receive and measure the variable through the first unit, while the control unit may be operatively coupled to the sensor unit and provide the value of the variable to the user. In the alternative and for both examples, the control member may include multiple sensor units and at least one control unit, where each of the sensor units may monitor the same variable or different variables, where at least one of the sensor units may also receive and monitor the variable through the first unit, and where the control unit may operatively couple with the sensor units and provide the value of the variable to the user.

In another aspect of the present invention, a pelvic exercise system may measure at least one variable which represents an extent of bending of at least a part of the system effected by at least a portion of the exercising standard pelvic structure and for providing a value of the variable to an user.

In one exemplary embodiment of this aspect of the invention, a system may include at least one standard body member and at least one control member including at least one sensor unit and at least one control unit. In one example, the sensor unit may measure force (or pressure) bending at least a part of the first unit, and the control unit may be operatively coupled to the sensor unit and provide the value of the bending in terms of such bending force (or pressure) to the user. In another example, the sensor unit may measure forces (or pressures) applied to different parts of the first unit and bending at least one of the parts and assess therefrom bending force (or pressure), while the control unit may be operatively coupled to the sensor unit and provide the value of the bending in terms of the bending force (or pressure) to the user. In another example, the sensor unit may measure displacement along the first unit effecting the bending, while the control unit may be operatively coupled to the sensor unit and provide the value of the bending in terms of the displacement to the user. In another example, the sensor unit may measure at least one angle along the first unit resulting from such bending, while the control unit may operatively couple with the sensor unit and provide the value of the bending in terms of the angle to the user. In another example, the sensor unit may monitor at least one curvature of the first unit resulting from the bending, and the control unit may be operatively coupled to the sensor unit and provide the value of such bending in terms of the curvature to the user. In another example, the sensor unit may measure deformation of the first unit resulting from the bending, while the control unit may be operatively coupled to the sensor unit and provide the value of such bending in terms of the deformation to the user.

In another exemplary embodiment of this aspect of the invention, a system may include at least one standard body member and at least one control member. In one example, the control member may include a single sensor unit and at least one control unit. The sensor unit may couple with the flexible part of the first unit and measure at least one variable effected by (or effecting) the bending of such a flexible part, and the control unit may operatively couple with the sensor unit and provide the value of the bending in terms of the variable. In another example, the control member may have multiple sensor units and at least one control unit. At least one of the sensor units may couple with the flexible part of the first unit and move therewith in response to the bending, and the sensor units may monitor at least one variable effected by (or effecting) the bending, and the control unit may be operatively coupled to the sensor unit and provide the value of the bending in terms of the variable.

In another exemplary embodiment of this aspect of the invention, a system may include at least one standard body member and at least one control member. The body member may have a rigid part, and the control member may include at least one sensor unit and at least one control unit. The sensor unit may be coupled to the rigid part of the first unit and measure at least one variable effected by (or effecting) the bending, and the control unit may be operatively coupled to the sensor unit and provide the value of the bending in terms of the variable.

In another aspect of the present invention, a pelvic exercise system may measure at least one variable representing physiologic state of at least a portion of the exercising standard pelvic structure of an user through monitoring a change in a variable electrical resistance of at least a part thereof in response to a value of the variable and for providing the value to the user.

In one exemplary embodiment of this aspect of the invention, a system may include at least one standard body member and at least one control member including at least one sensor unit and at least one control unit. In one example, the sensor unit may form therein at least one mobile part and at least one stationary part, define such electrical resistance based on a distance between the stationary and mobile parts, and then move the mobile part while changing the electrical resistance in response to the variable of the exercising structure. In another example, the sensor unit may also define at least one deformable part, define the electrical resistance based upon a dimension of the deformable part, and deform the part while changing the electrical resistance in response to the variable of the exercising structure. In another example, the sensor unit may include at least one conductive foam, define such electrical resistance based upon a dimension of the foam, and deform the foam while changing such electrical resistance in response to the variable of the exercising structure. For all of such examples, the control unit may operatively couple with the sensor unit and provide the value of the variable to the user.

In another exemplary embodiment of this aspect of the invention, a system may include at least one standard body member and at least one control member including at least one sensor unit and at least one control unit. In one example, the sensor unit may vary the electrical resistance in response to the exercising pelvic structure, while the control unit may be operatively coupled to the sensor unit and provide the value of the variable in terms of such resistance to the user. In another example, the sensor unit may effect deformation (or displacement) thereof while changing the electrical resistance in response to the exercising structure, while the control unit may operatively couple with the sensor unit and provide the value of the variable in terms of the deformation (or displacement) to the user. In another example, the sensor unit may exert force resisting the exercising structure while varying the electrical resistance, while the control unit may be operatively coupled to the sensor unit and provide the value of the variable in terms of the force to the user. In another example, such a sensor unit may vary the electrical resistance in response to at least one dynamic pattern of the exercising structure, and the control unit may operatively couple with the sensor unit and provide the value of the variable in terms of the dynamic pattern to the user.

In another exemplary embodiment of this aspect of the invention, a system may include at least one standard body member and at least one control member including at least one sensor unit and at least one control unit. In one example, the sensor unit may include at least one part capable of varying such electric resistance in response to the value of the variable. In another example, the sensor unit may include at least one conductive foam capable of varying its electric resistance in response to the value of the variable. In another example, the sensor unit may include multiple conductive foams each of which may extend circumferentially (or axially) and may vary the electrical resistance based upon its dimension in response to the variable of the exercising structure. In another example, the sensor unit may define multiple conductive foams at least one of each of which may extend circumferentially, at least one another of which may extent axially, and which may vary the electrical resistances based on their dimensions in response to the variable of the exercising structure. In another example, such a sensor unit may also define multiple conductive foams at least one of which may be disposed over an exterior of the first unit, at least one another of which may be disposed under such at least one of the conductive foam, and which may change the electrical resistances based upon their dimensions in response to the variable of the exercising structure. In each of these examples, the control unit may operatively couple with the sensor unit and provide the value of the variable to the user.

In another exemplary embodiment of this aspect of the invention, a system may include at least one standard body member and at least one control member including at least one sensor unit and at least one control unit. In one example, the sensor unit may include at least one part disposed over an exterior of the first unit and varying its electric resistance in response to the value of the variable. In another example, the sensor unit may include at least one part disposed under an exterior of the first unit and changing its electric resistance in response to the value of the variable which is transmitted thereto through the exterior. In each of these examples, the control unit may operatively couple with the sensor unit and provide the value of the variable to the user.

In another exemplary embodiment of this aspect of the invention, a system may include at least one standard body member and at least one control member. The body member may have at least one inflatable part, and the control member may have at least one sensor unit and at least one control unit. In one example, the sensor unit may include at least one part disposed on the inflatable part of the first unit and varying its electric resistance in response to the value of the variable while the inflatable part of the first unit inflates and deflates in response to the exercising structure. In another example, such a sensor unit may include at least one part disposed under (or inside) an exterior of the inflatable part of the first unit and varying its electric resistance in response to the value of the variable which may then be transmitted thereto through the inflatable part. In each of such examples, the control unit may operatively couple with the sensor unit and provide the value of the variable to the user.

In another aspect of the present invention, a pelvic exercise system may measure at least one variable representing physiologic state of at least a portion of the exercising standard pelvic structure of an user and resisting movement of at least a part of the system and provide a value of the variable to the user.

In one exemplary embodiment of this aspect of the invention, a system may include at least one standard body member and at least one control member including at least one sensor unit and at least one control unit. In one example, the first unit may vertically expand between multiple states so as to generate the movement, and the sensor unit may be disposed on (or inside) the first unit and measure the variable while the structure resists the movement of the first unit. In another example, the first unit may axially translate between multiple states in order to effect the movement, and such a sensor unit may be disposed on (or inside) the first unit and measure the variable while the structure resists the movement of the first unit. In another example, the first unit may angularly expand between multiple states in order to generate the movement, and the sensor unit may be disposed on (or inside) the first unit and measure the variable as the structure resists the movement of the first unit. In each of such examples, the control unit may be operatively coupled to the sensor unit and provide the value of the variable to the user.

In another exemplary embodiment of this aspect of the invention, a system may include at least one standard body member and at least one control member including at least one sensor unit and at least one control unit. In one example, the first unit may define a configuration and may be arranged to move out of the cavity due to its configuration when inserted into the cavity, and the sensor unit may be arranged to be disposed on (or inside) the first unit and measure the variable. In another example, the first unit may have a preset weight and may be arranged to move out of the cavity because of its weight when inserted into the cavity, and the sensor unit may be disposed on (or inside) the first unit and measure the variable. For each of these examples, the control unit may operatively couple with the sensor unit and provide the value of the variable to the user.

In another exemplary embodiment of this aspect of the invention, a system may include at least one standard body member and at least one control member. The body member may generate at least one of a vertical movement, axial movement, and rotational movement thereof, and the control member may have at least one sensor unit and at least one control unit. Such a first unit may move out of the internal cavity through the movement when inserted in the cavity. The sensor unit may be disposed on (or inside) the first unit and measure the variable, while the control unit may operatively couple with the sensor unit and provide the value of the variable to the user.

In another aspect of the present invention, a pelvic exercise system may measure at least one variable representing physiologic state of at least a portion of the exercising standard pelvic structure of an user exercising against a mechanical load posed by the system.

In one exemplary embodiment of this aspect of the invention, a system may include at least one body member and at least one control member. The body member may have at least one first unit and at least one load unit, the first unit may contact the portion of the structure when engaged therewith, and the load unit may be operatively coupled to the first unit and be also at least substantially resistant in nature. In one example, the load unit may include at least one resistant element which may change its mechanical resistance in response to an user input. In another example, the load unit may instead simultaneously receive a preset number of resistant elements each of which may then define a preset mechanical resistance, thereby exhibiting one of multiple different mechanical resistances. In another example, the load unit may releasably receive at least one resistant element which may have a preset mechanical resistance, thereby exhibiting one of multiple different mechanical resistances. In each of such examples, the control member may include at least one sensor unit and at least one control unit, where the sensor unit may monitor the variable, while the control unit may operatively couple with the sensor unit and provide a value of the variable to the user.

In another exemplary embodiment of this aspect of the invention, a system may include at least one body member and at least one control member. The body member may have at least one first unit and at least one load unit, where the first unit may contact the portion of the structure when engaged therewith, while the load unit may operatively couple with the first unit and to be at least substantially viscous in nature. In one example, the load unit may include at least one viscous element which may vary its viscosity in response to an user input. In another example, the load unit may include a preset number of viscous elements each defining a preset viscosity simultaneously, thereby exhibiting one of different viscosities. In another example, such a load unit may releasably receive at least one viscous element with a preset viscosity, thereby exhibiting one of multiple different mechanical resistances. In each of such examples, the control member may have at least one sensor unit and at least one control unit, where the sensor unit may monitor the variable, while the control unit may be operatively coupled to the sensor unit and provide a value of the variable to the user.

In another exemplary embodiment of this aspect of the invention, a system may include at least one body member and at least one control member. The body member may have at least one first unit and multiple load units, where the first unit may contact such a portion of the structure when engaged therewith. In one example, such load units may be operatively coupled to the first unit, to couple with each other in a series (or parallel) arrangement, and to be resistant elements. In another example, the load units may be operatively coupled to the first unit, couple with each other in a series (or parallel) arrangement, and to be viscous elements. In another example, the load units may operatively couple with the first unit, and couple with each other in a series (or parallel) arrangement, where at least one of such load units may be a resistant element, while at least another of the load units may then be a viscous element. In another example, such load units may be operatively coupled to the first unit, and couple with each other in a series (or parallel) arrangement, where at least one of the load units may be an inductive element, and the rest of the load units may be a resistant or viscous element. In each of these examples, the control member may include at least one sensor unit and at least one control unit, where the sensor unit may monitor the variable, while the control unit may be operatively coupled to the sensor unit and provide a value of the variable to the user.

In another aspect of the present invention, a pelvic exercise system may measure at least one variable representing physiologic state of at least a portion of the exercising standard pelvic structure of an user.

In one exemplary embodiment of such an aspect of the invention, a system may have at least one standard body member and at least one control member. In one example, the control member may include a single sensor unit and at least one control unit, where the sensor unit may measure multiple values of a single variable in multiple portions of the structure as the user maintains a preset posture of the structure, and where the control unit may be operatively coupled to the sensor unit, determine a configuration of the structure in the posture based upon the values, and provide information regarding the configuration to the user. In another example, the control member may have multiple sensor units and at least one control unit, where such sensor units may measure multiple values of the variable in multiple portions of the structure, where the user may maintain a preset posture of the structure, and where the control unit may be operatively coupled to the sensor units, assess a configuration of the structure in the posture based upon the values, and provide information as to the configuration to the user. In another example, the control member may have multiple sensor units and at least one control unit, where such sensor units may measure multiple values of the variables in multiple portions of the structure, where the user may maintain a preset posture of the structure, and where the control unit may be operatively coupled to the sensor units, assess a configuration of the structure in the posture based upon the values, and provide the user with information as to the configuration. For each of the examples, the control unit may provide the user with the values of the variables which are measured in such portions along with the information as to the configuration.

In another exemplary embodiment of this aspect of the invention, a system may include at least one standard body member and at least one control member. The control member may include at least one sensor unit and at least one control unit, where the sensor unit may monitor values of the variable in multiple portions of the pelvic structure as the user varies a posture of the structure, and where the control unit may be operatively coupled to the sensor unit, assess a configuration of the structure in each of the postures based on the values, construct movement of the structure based upon temporal changes in the postures, and provide the user with information regarding the movement.

In another aspect of the present invention, a pelvic exercise system may measure at least one variable representing physiologic state of at least a portion of the exercising standard pelvic structure of an user and for providing the user with at least one feedback signal.

In one exemplary embodiment of this aspect of the invention, a system may include at least one standard body member and at least one control member. The control member may include at least one sensor unit, at least one control unit, and at least one audio output unit. The sensor unit may measure the variable, and the audio output unit may operatively couple with the sensor unit and generate and play the feedback signal. In one example, the signal may be arranged to be an audible signal reflecting dynamic pattern of the variable. In another example, the signal may be arranged to be audible and to define an action basis (or a content basis) determined by the variable. In yet another example, such a signal may be arranged to be audible and to reflect comparison of the variable with a reference of the variable.

In another exemplary embodiment of this aspect of the invention, a system may include at least one standard body member and at least one control member. The control member may include at least one sensor unit, at least one control unit, and at least one visual output unit, where such a sensor unit may measure the variable, and where the visual output unit may be operatively coupled to the sensor unit and generate and display the feedback signal. In one example, such a signal may be arranged to be a visual signal which reflects dynamic pattern of the variable. In another example, the signal may be arranged to be visual and to include monochromic and/or color images whose intensities may also be determined by the variable. In another example, such a signal may be arranged to be visual and to include monochromic and/or color images reflecting the variable. In another example, the signal may be arranged to be visual and to reflect comparison of the variable with a reference of the variable.

In another exemplary embodiment of this aspect of the invention, a system may include at least one standard body member and at least one control member. The control member may include at least one sensor unit, at least one control unit, and at least one tactile output unit, where such a sensor unit may measure the variable, while the tactile output unit may operatively couple with the sensor unit and generate and deliver to the user the feedback signal. In one example, the signal may be arranged to be a tactile signal reflecting dynamic pattern of the variable. In another example, such a signal may be arranged to be tactile and to represent at least one movement of at least a part of the system of which an intensity is determined by the variable. In another example, the signal may be arranged to be tactile and to reflect comparison of the variable with a reference of the variable.

In another exemplary embodiment of this aspect of the invention, a system may include at least one standard body member and at least one control member. The control member may include at least one sensor unit, at least one control unit, and at least one output input. The sensor unit may measure the variable, and the output unit may operatively couple with the sensor unit and generate and display the feedback signal. In one example, such a signal may be an image reflecting the variable. In another example, the signal may be an image reflecting comparison of the variable with a reference thereof.

In another example, the signal may be an image describing a configuration of the first unit. In another example, the signal may be an image reflecting a position and/or configuration of the sensor (or first) unit. In another example, the signal may be an image displaying a static configuration of the structure which is superposed by another image reflecting a position and/or a configuration of the sensor (or first) unit. In yet another example, the signal may be an image displaying a dynamic configuration of the structure which is superposed by another image reflecting a position and/or a configuration of the sensor (or first) unit.

In another aspect of the present invention, a pelvic exercise system may measure at least one variable representing physiologic state of at least a portion of the exercising standard pelvic structure of an user and for providing a value of the variable to the user.

In one exemplary embodiment of this aspect of the invention, a system may include at least one body member and at least one control member. The body member may have at least one first unit and at least one second unit, where the first unit may contact such a portion of the pelvic structure when engaged therewith and where the second unit may form a grip for the user. The control member may include at least one sensor unit and at least one control unit, where the sensor unit may be included in the first unit and measure the variable, while the control unit may be incorporated in the body member, may be operatively coupled to the sensor unit, and may provide the value of the variable to the user.

In another exemplary embodiment of this aspect of the invention, a system may include at least one body member and at least one control member. The body member may have at least one first unit and at least one second unit, where the first unit may contact the portion of the pelvic structure when engaged therewith and where the second unit may form a grip for the user. The control member may include at least one sensor unit and at least one control unit, where the sensor unit may be included in the first unit and measure the variable, while the control unit may not be included in the body member, may be operatively coupled to the sensor unit through wire (or wirelessly), and may provide the value of the variable to the user.

In another exemplary embodiment of this aspect of the invention, a system may include at least one body member and at least one control member. The body member may have at least one first unit and at least one second unit, where the first unit may contact the portion of the pelvic structure when engaged therewith, while the second unit may define a grip for the user. In one example, the control member may have at least one sensor unit, at least one control unit, and at least one output unit. The sensor unit may be included into the first unit for monitoring the variable, while the control unit may be incorporated into (or away from) the body member and provide the value of the variable to the user. The output unit may be incorporated in the second unit and play (or display) at least one signal which reflects the value of such a variable. In another example, the control member may include at least one sensor unit and at least one control unit. The sensor unit may be included in the first unit and measure the variable. The control unit may be incorporated into (or away from) the body member and provide the value of the variable to the user, and operatively coupled to at least one output unit of an external device for transmitting at least one signal reflecting the variable to the external device through wire (or wirelessly), thereby playing (or displaying) the signal by the output unit of the external device.

In another aspect of the present invention, a pelvic exercise system may measure at least one variable representing physiologic state of at least a portion of the exercising standard pelvic structure of an user.

In one exemplary embodiment of this aspect of the invention, a system may include at least one standard body member and at least one control member. In one example, such a control member may include at least one sensor unit and at least one control unit. The sensor unit may monitor the variable, while the control unit may be operatively coupled to the sensor unit and provide the user with a value the variable, thereby allowing the user to monitor the physiologic state during exercising the structure. In another example, the control member may include at least one sensor unit and at least one control unit. The sensor unit may measure at least two of the variables when the user is in a preset posture of the structure. The control unit may operatively couple with the sensor unit, assess a configuration of the structure in the posture, and generate and display at least one image which may reflect such a configuration, thereby allowing the user to monitor such a configuration within the posture. In another example, the control member may include at least one sensor unit and at least one control unit, where the sensor unit may measure multiple the variables in multiple postures of the structure, and where the control unit may be operatively coupled to the sensor unit, assess a configuration of such a structure in each of the postures, and generate and display at least one image of movement reflecting changes in the configurations in response to changes in the postures, thereby allowing the user to monitor the movement.

In another exemplary embodiment of this aspect of the invention, a system may include at least one standard body member and at least one control member. The control member may include at least one sensor unit, at least one stimulation unit, and at least one control unit. Such a stimulation unit may supply electric and/or mechanical stimulation to the portion of the structure. Such a sensor unit may measure the variable, while the control unit may operatively couple with the stimulation and/or sensor units and then provide a value of the variable to the user, thereby allowing the user to monitor such a physiologic state during exercising the structure.

In another exemplary embodiment of this aspect of the invention, a system may include at least one standard body member and at least one control member. The control member may include at least one sensor unit and at least one control unit. The sensor unit may measure the variable, whereas the control unit may be operatively coupled to the sensor unit and play an audio feedback signal and/or a video signal reflecting the variable, thereby allowing the user to monitor the physiologic state during exercising the pelvic structure at least one of audibly and visually.

In another exemplary embodiment of this aspect of the invention, a system may include at least one standard body member and at least one control member. The control member may include at least one sensor unit and at least one control unit, where the control unit may play an audio signal and/or a video signal, where the sensor unit may measure the variable, while the control unit may operatively couple with the sensor unit and provide a value of the variable to the user, thereby allowing the user to monitor coincidence and discrepancy between such signals and the value of the variable.

Embodiments of such apparatus aspects of the present invention may include one or more of the following features.

The body member may also define a second unit for providing a grip to the user. The sensor unit may measure the variable in any direction with respect to a longitudinal axis of the body member. The sensor unit may measure an absolute value of the variable, a relative (or normalized) value of the variable with respect to a preset value thereof, and so on. The sensor unit may measure the variable after adjusting a baseline thereof. Such a variable may define a dynamic pattern which may include a temporal pattern and a spatial pattern, while the sensor unit may measure (or monitor) at least one of the temporal and spatial patterns, where the temporal pattern may be an instantaneous value of such a variable, its time-varying value, its time-averaged value, its average weighted by a preset weighting function, its peak value, its time derivative, its integration over time, and the like, and where the spatial pattern may be a localized value, distribution of multiple variables, its space averaged value, its global or local peak, its derivative along a preset direction, its integration over space, and the like.

The control member may obtain one of the variables and derive based thereupon at least one another of the variables. The control member may include any number of the sensor units which may be disposed in any arrangements and each of which may measure any of the variables. The sensor unit may monitor the variable while directly contacting the structure and/or through at least one article capable of transmitting the variable to the sensor therethrough, where such a transmitting article may be air, gas, fluid, and/or solid. The control member may instead have at least two of the sensor units which may measure the same variable in different parts of the body member, and the control unit may be operatively coupled to the sensor units and provide values of the variable in the different parts to the user. The control member may instead have at least two of the sensor units which may measure different variables in a preset part of the body member, while the control unit may operatively couple with the sensor units and provide values of the variables in the part to the user. The control member may instead have at least two of the sensor units which may measure different variables in different parts of the body member, while the control unit may be operatively coupled to the sensor units and provide values of the variables to the user.

Such bending may be initiated by the portion of the structure, and the sensor unit may measure the variable effecting (or effected) by such bending, where the variable may also define the dynamic pattern. Such bending may be initiated by the system, and the sensor unit may measure the variable resisting such bending, where the variable may also define the dynamic pattern. The bending may be effected or may effect in any directions. The control member may assess the bending force, bending pressure, displacement by the bending, angle thereby, curvature thereby, deformation thereby from at least one another thereof, and the like.

The sensor unit may include any number of the variable resistance parts. The sensor unit may include multiple variable resistance parts which may be disposed in any arrangements over (or in) the sensor unit. The sensor unit may also include multiple variable resistance parts which may measure the same or different variables. The sensor unit may include multiple variable resistance parts, while the control unit may receive signals simultaneously or one at a time from the variable resistance parts. The variable resistance part may exhibit elastic and/or viscous responses to the exercising structure.

The movement of the first unit may be generated by supplying and withdrawing air, gas, fluid, and/or water thereto and therefrom. The movement of the first unit may be effected by at least a part of the first unit which may be actuated by an actuator member. The movement of the first unit may be synchronized with audio signals and/or visual signals provided by the system. The movement of the first unit may correspond to a movement of only a part of the first unit or a movement of an entire part of the first unit. The system may further move not only the first unit but also at least a part of the body member for the movement.

The load unit may also include a mass element with a non-negligible mass, where such a mass element may be the part of the first or sensor unit. Such a control member may select suitable values of the resistance, viscosity, and/or mass based on a second-order differential equation of the system. The variable and/or value may be in an analog or digital format.

The system may play sound and/or display an image of the variable and/or value with internal audio, visual, and/or audiovisual input and/or output units or with external audio, visual or audiovisual input and/or output unit of the external devices. The sound and/or image may further be generated in response to the value and/or variable effected (or initiated) by the user or may be generated to effect the value and/or variable by the user.

Such a portion of the pelvic structure may be a clitoris of the entry, a G spot on the wall, other portions of the wall, and the like. The sensor unit may be defined in its head, trunk, and/or base of the first unit. The body member may include multiple sensor units which may be identical, disposed close to each other, disposed apart from each other, and the like. The part may be designed to engage with the clitoris, G spot, and the like.

Such dynamic pattern of the variable may be at least one of its temporal pattern and its spatial pattern, where the temporal pattern may be at least one of a duration of the variable, its frequency, and its temporal sequence, and where the spatial pattern may include at least one of an amplitude of the variable, and its direction. The dynamic pattern may be at least one of a frequency of the variable, its temporal rate of change (or its temporal differentiation), its displacement (or its integral over time) caused thereby, and a compound value obtained by at least one of mathematical manipulation of at least one thereof.

In another aspect, a method may be provided for monitoring at least one variable representing physiologic state of at least a portion of the exercising standard pelvic structure of an user by at least one sensor unit of a pelvic exercise system.

In one exemplary aspect of such an aspect of the present invention, a method may include the steps of: operatively coupling the sensor unit with the portion of the structure; exercising the muscles of the structure; and measuring the variable which may be normal force applied onto at least a part of the system, bending force applied to the part, axial force pulling (or pushing) the part into (or from) the internal cavity of the structure, torque applied around the part, velocity of the part, acceleration of the part, displacement of the part, contact between such a part and such a portion, a dimension of such a portion, contraction and relaxation of the portion, a duration of at least one of such variables, and/or a frequency of at least one of the variables. The above measuring may be replaced by another step of: measuring the variable which may be normal force applied onto or by such a portion of the structure, bending force applied to (or by) the portion, axial force resisting movement of the part into (or out of) the internal cavity of the pelvic structure, torque applied around such a portion, velocity of the portion, acceleration of such a portion, displacement of the portion, contact between such a part and such a portion, contraction and relaxation of the portion, a duration of at least one of the variables, and/or a frequency of at least one of the variables.

In another exemplary aspect of such an aspect of the present invention, a method may include the steps of: operatively coupling such a sensor unit with the portion of the structure; exercising the structure; and measuring the variable by the sensor unit, thereby monitoring the state of the structure during the exercising. The exercising may be replaced by the steps of: generating movement of the part of the system; and exercising the structure against the movement; and

In another exemplary aspect of such an aspect of the present invention, a method may include the steps of: operatively coupling such a sensor unit with the portion of the structure; exercising the structure while maintaining or keeping a preset posture of the structure; and measuring the variable, thereby monitoring the state of the structure in the posture. The above exercising and measuring may be replaced by the steps of: exercising the structure while changing postures of the structure; and measuring the variable, thereby monitoring the state of the structure during the exercising in each of the postures. The above exercising and measuring may be replaced by the steps of: exercising the structure while contracting other muscles of an abdomen, a back, a waist, a leg, and/or a thigh of the user and while affecting the variable; and measuring the variable through such a sensor unit, thereby monitoring the state of the structure during the exercising of the muscles of the pelvic structure and the other muscles.

In another aspect, a method may be provided for monitoring at least one variable representing physiologic state of at least a portion of the exercising standard pelvic structure of an user by multiple sensor units of a pelvic exercise system.

In one exemplary aspect of such an aspect of the present invention, a method may include the steps of: disposing at least two of the sensor units in a single part of the system; operatively coupling at least two of the sensor units with a single portion of the structure; exercising the muscles of such a structure; and measuring different variables using such at least two of the sensor units during the exercising, thereby obtaining spatial distribution of each of the variables.

In another exemplary aspect of such an aspect of the present invention, a method may include the steps of: disposing at least two of the sensor units in different parts of such a system; operatively coupling at least two of the sensor units with different portions of the pelvic structure; exercising the muscles of the structure; and measuring a single variable using such at least two of the sensor units during the exercising, thereby obtaining spatial distribution of the variable. The measuring step may be replaced by the step of: measuring different variables by such at least two of the sensor units during the exercising, thereby obtaining spatial distribution of each of the variables.

In another exemplary aspect of such an aspect of the present invention, a method may include the steps of: disposing the sensor units in the system; operatively coupling the sensor units with the portion of the structure; exercising the muscles of the structure; and measuring the variable using at least one of the sensor units over time, thereby obtaining temporal distribution of the variable.

In another exemplary aspect of such an aspect of the present invention, a method may include the steps of: disposing the sensor units in the system; operatively coupling the sensor units with the portion of the structure; exercising the muscles of the structure; measuring the variable; and moving at least one of the sensor units with respect to the structure during the measuring, thereby obtaining spatial distribution of the variable.

In another aspect, a method may be provided for monitoring at least one variable representing physiologic state of at least a portion of the exercising standard pelvic structure of an user by at least one sensor unit disposed in one of multiple preset parts (or areas) of a pelvic exercise system.

In one exemplary aspect of such an aspect of the present invention, a method may include the steps of: disposing the sensor unit across a substantial part (or area) (or preset part or area) of the system; operatively coupling the sensor unit with the portion of the structure; exercising the muscles of the pelvic structure; and measuring the variable by the sensor unit during such exercising, thereby obtaining a value representing the variable across the part (or area). The disposing may be replaced by the step of: disposing the sensor unit along at least a part of a circumference around the system or by the step of: disposing the sensor unit in a part (or area) parallel to a longitudinal axis of the system;

In another exemplary aspect of such an aspect of the present invention, a method may include the steps of: disposing multiple the sensor units across substantial (or preset) parts (or areas) of the system; operatively coupling such sensor units to a single portion (or multiple different portions) of the structure; exercising the muscles of the structure; and measuring a single variable by the sensor units during the exercising, thereby obtaining a value representing the variable across the parts (or areas). The disposing may also be replaced by the step of: disposing the sensor unit along at least a part of a circumference about the system or by the step of: disposing the sensor unit in a part (or area) parallel to a longitudinal axis of the system. The measuring may be replaced by the step of: measuring multiple different variables with the sensor units during the exercising, thereby obtaining multiple values which represent a spatial distribution of the variable across the parts (or areas).

In another aspect, a method may be provided for contacting at least a portion of the exercising standard pelvic structure of an user and monitoring at least one variable which represents physiologic state of the portion of the structure by at least one sensor unit of a pelvic exercise system.

In one exemplary aspect of such an aspect of the present invention, a method may include the steps of: inserting the sensor unit into the internal cavity; operatively coupling the sensor unit with the portion of the structure; exercising the muscles of the structure; and measuring the variable using the sensor unit, thereby monitoring the state of the structure during the exercising.

In another exemplary aspect of such an aspect of the present invention, a method may include the steps of: inserting the sensor unit into the internal cavity; inflating at least a part of the sensor unit, thereby fitting such a sensor unit unto the structure; exercising the muscles of the structure; and then measuring the variable using the sensor unit, thereby monitoring the state of the structure during such exercising.

In another exemplary aspect of such an aspect of the present invention, a method may include the steps of: inserting at least a part of the system into the internal cavity; disposing the sensor unit in the part of the system; inflating (or moving) the part, thereby fitting the sensor unit onto the structure; exercising the muscles of the structure; and then measuring the variable with the sensor unit, thereby monitoring the state of the structure during the exercising.

In another exemplary aspect of such an aspect of the present invention, a method may include the steps of: inserting at least a part of the system into the internal cavity; operatively coupling the part with the sensor unit; exercising such muscles of the structure; transmitting the variable to the sensor unit through the part; and measuring the variable using the sensor unit, thereby monitoring the state of the structure during the exercising.

In another aspect, a method may be provided for monitoring at least one variable representing an extent of bending of at least a part of a pelvic exercise system effected by at least a portion of the exercising standard pelvic structure of an user.

In one exemplary aspect of such an aspect of the present invention, a method may include the steps of: arranging the part of the system to be flexible; incorporating a single sensor unit in the part; operatively coupling the part with the portion of the structure; exercising the muscles of the structure, thereby bending the part; and measuring the variable and extent by the sensor unit, thereby monitoring physiologic state of the structure during the exercising.

In another exemplary aspect of such an aspect of the present invention, a method may include the steps of: arranging such a part of the system to be flexible; incorporating multiple sensor units in opposite ends of the part; operatively coupling the part with the portion of the structure; exercising the muscles of the pelvic structure, thereby bending the part; measuring such variables in the ends; and assessing the extent by the variables, thereby monitoring physiologic state of the structure during the exercising.

In another exemplary aspect of such an aspect of the present invention, a method may include the steps of: incorporating a single sensor unit in the part; operatively coupling the part to the portion of the structure; exercising such muscles while applying force onto the part; measuring the force with the sensor unit; and expressing such an extent in terms of the force. The exercising, measuring, and expressing may be replaced by the steps of: exercising such muscles of the structure while applying force onto the part; measuring the force using the sensor unit; and assessing the variable and extent from the force. The above exercising, measuring, and expressing may also be replaced by the steps of: exercising the muscles of such a structure while effecting displacement of the part; measuring the displacement using the sensor unit; and then expressing the extent in terms of the displacement. The exercising, measuring, and expressing may also be replaced by the steps of: exercising the muscles of the structure while effecting displacement of the part; measuring such displacement by the sensor unit; and assessing the variable and extent from the displacement. Such exercising, measuring, and expressing may be replaced by the steps of: exercising the muscles of the structure while varying curvature around the part; measuring the curvature (or change thereof) by the sensor unit; and then expressing the extent in terms of the curvature (or change thereof). The exercising, measuring, and expressing may be replaced by the steps of: exercising such muscles of the structure while varying curvature around the part; measuring the curvature (or change thereof) by the sensor unit; and then assessing the variable and extent from the curvature (or change thereof).

In another aspect, a method may be provided for monitoring at least one variable representing physiologic state of at least a portion of the exercising-standard pelvic structure of an user by at least one article with variable resistance of a pelvic exercise system.

In one exemplary aspect of such an aspect of the present invention, a method may include the steps of: operatively coupling the article with the portion of the structure; exercising such muscles of the structure while varying configuration of such an article; changing the resistance of the article in response to the varying; and measuring the variable from the resistance, thereby monitoring the state of the structure during the exercising.

In another exemplary aspect of such an aspect of the present invention, a method may include the steps of: operatively coupling the article with the portion of the structure; exercising the muscles of the structure while deforming the article; changing the resistance of the article in response to the deforming; and measuring the variable from the resistance, thereby monitoring the state of the pelvic structure during the exercising.

In another exemplary aspect of such an aspect of the present invention, a method may include the steps of: forming such an article from a conductive foam; operatively coupling the article with the portion of the structure; exercising the muscles of the structure while squeezing the article; changing the resistance of the article in response to the squeezing; and then measuring the variable from such resistance, thereby monitoring the state of the structure during the exercising.

In another exemplary aspect of such an aspect of the present invention, a method may include the steps of: operatively coupling the article with the portion of the structure; exercising the muscles of the structure; varying the resistance of the article during the exercising; and measuring the variable from the resistance, thereby monitoring the state of the structure during the exercising. Such varying may be replaced by the step of: varying configuration of the article during such exercising; and then changing the resistance of the article in response to the varying. The varying may be replaced by the step of: displacing (or moving) the article during the exercising; and then changing such resistance of the article in response to the displacing.

In another exemplary aspect of such an aspect of the present invention, a method may include the steps of: inserting the article into the internal cavity; operatively coupling the article with the portion of the structure; exercising the muscles of the structure; and measuring such a variable by the article, thereby monitoring the state of the structure during the exercising.

In another exemplary aspect of such an aspect of the present invention, a method may include the steps of: inserting the article into the internal cavity; inflating at least a part of the article, thereby fitting the sensor unit unto the structure; exercising the muscles of the structure; and measuring such a variable by the article, thereby monitoring the state of the structure during the exercising.

In another exemplary aspect of such an aspect of the present invention, a method may include the steps of: inserting at least a part of the system into the internal cavity; disposing the article in the part of the system; inflating or moving the part, thereby fitting the article onto the structure; exercising the muscles of the structure; and measuring the variable by the article, thereby monitoring the state of the structure during the exercising.

In another exemplary aspect of such an aspect of the present invention, a method may include the steps of: inserting at least a part of the system into the internal cavity; operatively coupling the part with the article; exercising the muscles of the structure; transmitting the variable to the article through the part; and measuring the variable by the article, thereby monitoring the state of the structure during the exercising.

In another exemplary aspect of such an aspect of the present invention, a method may include the steps of: forming the article from conductive foam; aligning the article along a circumference of the system; operatively coupling the article with the portion of the pelvic structure; exercising the muscles of the structure while squeezing the article; changing such resistance of the article in response to the squeezing; and measuring the variable from the resistance, thereby monitoring the state of the pelvic structure during the exercising. The aligning may be replaced by the step of: aligning the article along a longitudinal axis of the system or by the step of: helically aligning the article around the system.

In another aspect, a method may be provided for monitoring at least one variable representing physiologic state of at least a portion of the exercising standard pelvic structure of an user by multiple conductive foams for a pelvic exercise system.

In one exemplary aspect of such an aspect of the present invention, a method may include the steps of: disposing at least two conductive foams in a single part of the system; operatively coupling the conductive foams with a single portion of the structure; exercising such muscles of the structure; and measuring multiple variables using the conductive foams during the exercising, thereby obtaining spatial distribution of each of the variables.

In another exemplary aspect of such an aspect of the present invention, a method may include the steps of: disposing at least two conductive foams in different parts of such a system; operatively coupling the conductive foams with different portions of the structure; exercising the muscles of the structure; and measuring a single variable using the conductive foams during the exercising, thereby obtaining spatial distribution of the variable. The measuring may be replaced by the step of: measuring multiple variables by the conductive foams during the exercising, thereby obtaining spatial distribution of each of the variables.

In another exemplary aspect of such an aspect of the present invention, a method may include the steps of: disposing the conductive foams in the system; operatively coupling the conductive foams with the portion of the structure; exercising such muscles; and measuring the variable by at least one of the conductive foams over time, thereby obtaining temporal distribution of the variable.

In another exemplary aspect of such an aspect of the present invention, a method may include the steps of: disposing at least two conductive foams in the system; operatively coupling such foams with the portion of the structure; exercising the muscles of the structure; measuring the variable; and moving at least one of such conductive foams relative to the structure during the measuring, thereby obtaining spatial distribution of the variable.

In another aspect, a method may be provided for contacting at least a portion of the exercising standard pelvic structure of an user in order to monitor at least one variable representing physiologic state of the portion of the structure by at least one sensor unit of a pelvic exercise system.

In one exemplary aspect of such an aspect of the present invention, a method may include the steps of: including at least one movable part in the system; disposing the part of the system into such an internal cavity; exercising the muscles of the structure; vertically expanding such a part toward the muscles during the exercising; and measuring the variable using the sensor unit, thereby monitoring the state of the structure during the exercising. The expanding may be replaced by the step of: axially extending the part toward the muscles during the exercising or by the step of: angularly rotating such a part toward the muscles during the exercising.

In another exemplary aspect of such an aspect of the present invention, a method may include the steps of: including into the system at least one part movable by pressure by air, gas, and/or fluid; disposing the part of the system into such an internal cavity; exercising the muscles of the structure; moving the part toward the muscles during the exercising; and measuring the variable by the sensor unit, thereby monitoring the state of the pelvic structure during the exercising. The including may be replaced by the step of: including into the system at least one part movable by mechanical actuation thereof or by the step of: including in the system at least one part defining at least one screw thereon and movable by rotation of the screw.

In another exemplary aspect of such an aspect of the present invention, a method may include the steps of: including in the system at least one part with one of a preset configuration and a weight; inserting the part of the system into the internal cavity; moving the part toward the opening due to the one of the configuration and weight; exercising the muscles of the pelvic structure while resisting the moving; and measuring the variable using the sensor unit, thereby monitoring the state of the structure during the exercising.

In another aspect, a method may be provided for monitoring at least one variable representing physiologic state of at least a portion of the standard pelvic structure of an user exercising against at least one load unit of a pelvic exercise system.

In one exemplary aspect of such an aspect of the present invention, a method may include the steps of: operatively coupling the sensor unit with the portion of the structure; exercising the muscles of the structure against the load unit with a preset resistance; and measuring the variable through the sensor unit, thereby evaluating the muscles exercising against the preset resistance. The exercising and measuring may be replaced by the steps of: exercising the muscles of the structure against the load unit with a preset viscosity; and measuring the variable by the sensor unit, thereby evaluating the muscles exercising against the preset viscosity. The method may also include at least one of optional steps of: replacing the load unit with another unit with another resistance (or viscosity); adding to the load unit another load unit defining another resistance (or viscosity); and adjusting the resistance (or viscosity) of the load unit.

In another exemplary aspect of such an aspect of the present invention, a method may include the steps of: providing multiple load units each defining a preset resistance and/or viscosity; coupling the load units in a series arrangement and/or parallel arrangement; operatively coupling the sensor unit with the portion of the structure; exercising such muscles of the structure against the load units; and measuring the variable by the sensor unit, thereby evaluating the muscles exercising against the load units. The method may include at least one of the optional steps of: replacing at least one of the load units by another unit with another resistance (or viscosity); adding to such load units at least another load unit with another resistance (or viscosity); removing at least one of the load units from the rest of the load units; and adjusting the resistance (or viscosity) of at least one of the load units.

In another exemplary aspect of such an aspect of the present invention, a method may include the steps of: operatively coupling the sensor unit with such a portion of the structure; exercising the muscles of the structure against the load unit with a preset viscosity and/or resistance; measuring the variable through the sensor unit; and adjusting such viscosity and/or resistance of the load unit based upon the variable, thereby exercising the muscles against the adjusted load unit.

In another exemplary aspect of such an aspect of the present invention, a method may include the steps of: operatively coupling the sensor unit with the portion of the structure; providing multiple load units each defining a preset resistance and/or viscosity; coupling the load units in a series and/or parallel arrangement; exercising the muscles of such a structure against the load units; measuring the variable through the sensor unit; and adjusting such viscosity and/or resistance of at least one of the load units based on the variable, thereby exercising the muscles against the adjusted load units.

In another aspect, a method may also be provided for assessing at least one optimum posture by monitoring at least one variable representing physiologic state of at least a portion of the exercising standard pelvic structure of an user using at least one sensor unit of a pelvic exercise system.

In one exemplary aspect of such an aspect of the present invention, a method may include the steps of: operatively coupling the sensor unit with the portion of the structure; exercising the muscles of the structure while maintaining the posture of the structure and without contracting other muscles of the user; and measuring the variable by the sensor unit, thereby monitoring the state of the pelvic structure in the posture during the exercising.

In another exemplary aspect of such an aspect of the present invention, a method may include the steps of: operatively coupling the sensor unit with the portion of the pelvic structure; exercising the muscles of the structure while posing each of multiple the postures of the structure and without contracting other muscles of the user; and then measuring the variable with the sensor unit, thereby monitoring the state of the structure in each of the postures during the exercising and selecting one of the postures as the optimum one.

In another exemplary aspect of such an aspect of the present invention, a method may include the steps of: operatively coupling such a sensor unit with the portion of the structure; exercising the muscles of the structure while posing each of multiple the postures of the structure successively and without contracting other muscles of the user; and measuring the variable by the sensor unit, thereby monitoring the state of the pelvic structure in each of the postures during the exercising and selecting a preset series of the postures as the optimum ones.

In another exemplary aspect of such an aspect of the present invention, a method may include the steps of: operatively coupling the sensor unit with such a portion of the structure; exercising the muscles of the structure while maintaining the posture of the structure and while contracting at least one another muscle of the user; and measuring the variable by the sensor unit, thereby monitoring the state of the structure in the posture during the exercising and contracting.

In another exemplary aspect of such an aspect of the present invention, a method may include the steps of: operatively coupling such a sensor unit with the portion of the structure; exercising the muscles of the pelvic structure while posing each of multiple the postures of the structure and while contracting at least one another muscle of the user; and measuring the variable with the sensor unit, thereby monitoring the state of the structure in each of the postures during the exercising as well as contracting and thereby selecting one of the postures as the optimum one.

In another exemplary aspect of such an aspect of the present invention, a method may include the steps of: operatively coupling such a sensor unit with the portion of the structure; exercising the muscles of the structure while posing each of multiple the postures of the structure successively and while contracting at least one another muscle of the user; and measuring the variable with the sensor unit, thereby monitoring the state of the structure in each of the postures during the exercising as well as contracting and thereby selecting a preset series of the postures as the optimum ones.

In another exemplary aspect of such an aspect of the present invention, a method may include the steps of: operatively coupling such a sensor unit with the portion of the structure; exercising the muscles while posing multiple the postures of the structure; and measuring such a variable in multiple different portions of the structure by the sensor unit during the posing, thereby monitoring the state in each of the postures during the exercising and selecting one of the postures as the optimum one. The measuring may be replaced by the step of: measuring multiple variables in each of multiple different portions of the structure by the sensor unit during the posing, thereby monitoring the state in each of the postures during the exercising and selecting one of the postures as the optimum one.

In another exemplary aspect of such an aspect of the present invention, a method may include the steps of: operatively coupling multiple the sensor units with the portion of the structure; exercising the muscles while posing multiple the postures of the structure; and measuring the variable in multiple different portions of the structure by the sensor units during the posing, thereby monitoring the state in each of the postures during the exercising and selecting one of the postures as the optimum one. The measuring may also be replaced by the step of: measuring multiple variables in multiple different portions of the structure with the sensor units during the posing, thereby monitoring the state in the postures during the exercising and selecting one of the postures as the optimum one.

In another aspect, a method may be provided for monitoring at least one variable representing physiologic state of at least a portion of the exercising standard pelvic structure of an user by at least one sensor unit of a pelvic exercise system and for also providing at least one feedback signal for the variable.

In one exemplary aspect of such an aspect of the present invention, a method may include the steps of: operatively coupling the sensor unit with the portion of the structure; exercising the muscles of the structure; and then measuring the variable by the sensor unit while playing sounds which may reflect the variable, may include at least one content basis reflecting the variable, may include at least one content basis comparing the variable with a reference thereof or may include at least one content basis instructing the user as to the exercising.

In another exemplary aspect of such an aspect of the present invention, a method may include the steps of: operatively coupling such a sensor unit with the portion of the structure; exercising the muscles of the structure; and measuring the variable by the sensor unit while playing images which may reflect the variable, may display at least one content basis reflecting the variable, may display at least one content basis comparing the variable with a reference thereof or may display at least one content basis instructing the user as to the exercising.

In another exemplary aspect of such an aspect of the present invention, a method may include the steps of: operatively coupling such a sensor unit with the portion of the structure; exercising the muscles of the structure; and then measuring the variable by the sensor unit while generating tactile sensations which may reflect such a variable, may reflect at least one content basis of the variable or may reflect at least one content basis comparing the variable with a reference thereof.

In another exemplary aspect of such an aspect of the present invention, a method may include the steps of: operatively coupling such a sensor unit with the portion of the structure; exercising the muscles of the structure; and measuring the variable with the sensor unit while playing images of the sensor unit which may reflect disposition of the sensor unit, may reflect configuration thereof, may be superposed with the variable or may be compared with a reference thereof.

In another exemplary aspect of such an aspect of the present invention, a method may include the steps of: operatively coupling such a sensor unit with the portion of the structure; exercising the muscles of the structure; and measuring the variable with the sensor unit while playing images of the structure in a preset posture thereof, while playing images of such a structure changing with each of multiple postures thereof or while playing images of such a structure superposed with configuration or disposition of the sensor unit.

In another aspect, a method may be provided for monitoring at least one variable representing physiologic state of at least a portion of the exercising standard pelvic structure of an user by at least one sensor unit of a pelvic exercise system.

In one exemplary aspect of such an aspect of the present invention, a method may include the steps of: operatively coupling the sensor unit with the portion of the structure; exercising the muscles of the structure; and measuring the variable by such a sensor unit while providing the variable to the user, thereby allowing the user to monitor improvement in contraction of the muscles.

In another exemplary aspect of such an aspect of the present invention, a method may include the steps of: inserting the sensor unit into the internal cavity of the structure; moving the muscles to multiple portions the structure; and measuring such a variable by the sensor unit, thereby assessing a location of a preset portion of the structure providing a desired sensation to the user.

In another exemplary aspect of such an aspect of the present invention, a method may include the steps of: operatively coupling such a sensor unit with the portion of the structure; exercising the muscles of the structure while maintaining a posture of the structure; and measuring such a variable by the sensor unit, thereby monitoring the state of the structure in the posture during the exercising.

In another exemplary aspect of such an aspect of the present invention, a method may include the steps of: operatively coupling such a sensor unit with the portion of the structure; exercising the muscles of the structure while disposing the structure in each of multiple postures thereof; and then measuring the variable with the sensor unit, thereby assessing a desired posture from the postures.

In another exemplary aspect of such an aspect of the present invention, a method may include the steps of: operatively coupling such a sensor unit with the portion of the structure; exercising the muscles of the structure; and measuring the variable by the sensor unit while providing the user with the variable and playing sounds reflecting the variable, thereby allowing the user to audibly hear such a variable.

In another exemplary aspect of such an aspect of the present invention, a method may include the steps of: operatively coupling the sensor unit to the portion of the pelvic structure; playing preset sounds; exercising the muscles of the pelvic structure in response to the sounds; and measuring the variable by the sensor unit, thereby allowing the user to synchronize the exercising with the sounds.

In another exemplary aspect of such an aspect of the present invention, a method may include the steps of: operatively coupling such a sensor unit with the portion of the structure; exercising the muscles of the structure; and measuring the variable by the sensor unit while providing the user with the variable and displaying images reflecting the variable, thereby allowing the user to visually monitor the variable.

In another exemplary aspect of such an aspect of the present invention, a method may include the steps of: operatively coupling the sensor unit with the portion of the structure; displaying preset images; exercising the muscles of the structure in response to the images; and measuring the variable with the sensor unit, thereby allowing the user to synchronize the exercising with the images.

Embodiments of such method aspects of the present invention may include one or more of the following features.

The coupling may include one of the steps of: including a single sensor unit to the system; and including multiple sensor units into the system. The coupling may include one of the steps of: aligning the sensor unit parallel or perpendicular to a longitudinal axis of the system; extending the sensor unit along a circumference of the system; disposing the sensor unit at a preset angle with respect to the longitudinal axis; and disposing the sensor unit in a helical pattern around the system. Such coupling may include at least one of the steps of: directly contacting such a sensor unit with the portion of the structure; and coupling the sensor unit through at least one transmitting article to the portion.

The exercising may include at least one of the steps of: exercising the structure against such a part which is stationary; exercising against the structure which is mobile; and exercising against the part which is arranged to change its configuration. The exercising may instead include at least one of the steps of: contracting and relaxing the muscles of the structure while keeping the posture of the structure; contracting and relaxing the muscles of such a pelvic structure while varying the postures; contracting and relaxing the muscles of the structure without relaxing and contracting another portion of a body of the user; and contracting and relaxing the muscles of the structure while contracting and relaxing the muscles of such another portion. The exercising may instead include one of the steps of: performing such exercising in a pace selected by the user; conforming such exercising to an audible sound played by the system; and conforming the exercising to a visual image which may be displayed by the system. The exercising may include the above bending. The bending may be in any direction, and may define the dynamic pattern.

The disposing may include one of the steps of: incorporating the sensor units over (or under) at least substantially identical parts of the system; disposing the sensor units adjacent (or close) to each other; disposing at least two of the sensor units on opposite surfaces of the system; aligning the sensor units perpendicular to each other; and aligning such sensor units parallel to each other. Such disposing may include one of the steps of: incorporating the sensor units on an exterior of the system; incorporating the sensor units under the exterior; and disposing at least one of the sensor units on the exterior while at least another thereof under the exterior. The disposing may also include at least one of the steps of: extending at least one of the sensor units along the longitudinal axis of such a system; winding at least one of the sensor units around the circumference of such a system; and disposing at least one of the sensor units helically around the system.

The measuring may include one of the steps of: measuring multiple variables one at a time with multiple sensor units; measuring multiple variables simultaneously by multiple sensor units; measuring a single variable simultaneously by multiple sensor units disposed in multiple portions of the structure; and measuring the single variable one at a time by multiple sensor units disposed in multiple positions of the structure. The measuring may also include at least one of the steps of: maintaining the preset posture during the measuring; changing the postures (or generating movement of the pelvic structure) during the measuring; and contracting and/or relaxing other muscles of at least one of an abdomen, back, waist, leg, and thigh of the user. The measuring may include one of the steps of: contracting and relaxing the muscles of the structure while keeping the posture during the measuring; changing the posture without relaxing and contracting the muscles during the measuring; and contracting and relaxing the muscles while also changing the posture during the measuring. The measuring may also include the step of: measuring the variable in any direction with respect to a longitudinal axis of the system. The measuring may include at least one of the steps of: measuring an absolute value of the variable; and measuring a relative (or normalized) value of the variable with respect to its reference value. The measuring may include the step of: measuring the variable after adjusting a baseline of the sensor unit. The adjusting may include the steps of: disposing the sensor unit on an inflatable part of the system; inflating the inflatable part onto the structure; and measuring the variable. Such adjusting may include the steps of: inflating at least one inflatable part of the system onto the pelvic structure; operatively coupling the sensor unit to the part; and measuring the variable by the sensor unit through the inflatable part.

The measuring may include the step of: monitoring a dynamic pattern of such a variable which may include a temporal pattern and/or a spatial pattern. The monitoring may include at least one of the steps of: monitoring the temporal pattern which may include an instantaneous value of the variable, its time-averaged value, its time-varying value, its average weighted by a preset weighting function, its peak value, its time derivative, its integration over time, and the like; and monitoring the spatial pattern which may be at least one of a localized value, distribution of multiple variables, its space averaged value, its global or local peak, its derivative along a preset direction, its integration over space, and the like. The measuring may include the steps of: obtaining one of the variables; and deriving at least one another of the variables based upon the one of the variables. The disposing such a sensor unit may include the step of: using the variable resistance article(s) as the sensor unit(s). The measuring the variable may include the steps of: using the variable resistance article as such a sensor unit; and then measuring the variable based upon the resistance of the article. Such disposing the sensor unit may include the step of: using the conductive foam(s) as the sensor unit(s). The measuring the variable may include the step of: employing the conductive foam as the sensor unit; and measuring the variable based upon the resistance of the conductive foam. The method may include the step of: providing the system with a grip for the user.

Various product-by-process claims may be constructed by modifying the foregoing preambles or their modifications of the above system and/or method claims and by appending thereto the above bodies or their modifications of the above system and/or method claims. Such process claims may be arranged to include one or more of the aforementioned features of the above system and/or method claims of the present invention.

As used herein, a term “pelvic structure” refers to an anatomic structure of sexual organs of a female. Such a “pelvic structure” typically defines an entry and a wall, where the entry is arranged to define an orifice therethrough, and where the wall is arranged to include various muscles and to also define an internal cavity which is arranged to extend inwardly and to be also bound by the muscles.

As used herein, both of the terms “sensor unit” and “input unit” refer to those units of various pelvic exercise systems capable of receiving various variables of the pelvic structure (or user inputs) and/or monitoring dynamic pattern thereof. However, the “sensor unit” and the “input unit” are to be differentiated as follows within the scope of this invention. First, such a “sensor unit” is preferably disposed in an insertable part of a body member, while the “input unit” is generally disposed in a part of the body member of the system which is intended to not be inserted into the internal cavity of such a pelvic structure. Accordingly, the “sensor unit” generally receives the user inputs through various portions of the pelvic structure, whereas the “input unit” mainly receives the variables (or user inputs) through a hand or a finger of an user.

The terms “proximal” and “distal” will be used in a relative context. Throughout this invention, the term “proximal” is to be used to denote a direction toward a head of a body member of a system, whereas the term “distal” is to be used to represent an opposite direction toward an end of a handle of such a system. Accordingly, a “proximal” end and a “distal” end may be defined with respect to an entire pelvic exercise system or with respect to a specific member or unit thereof.

The terms “input unit” and “sensor unit” generally refer to identical or similar articles capable of monitoring various dynamic patterns of various user inputs applied thereto. Throughout this invention, however, the “input units” represent such articles incorporated into a handle part (or a second unit) of a body member of a pelvic relaxing system, while the “sensor units” denote such articles incorporated into an insertable part (or a first unit) of such a body member. Accordingly, any articles which may be used as the “input unit” may also be used as the “sensor unit” unless otherwise specified. In addition, the “input unit” may also be disposed in the insertable part of the body member, while the “sensor unit” may also be disposed in the handle part thereof when desired.

As used herein, a “dynamic pattern” refers to a temporal pattern as well as a spatial pattern of a variable (i.e., a “pelvic variable”), of an user input, and/or of a sensing signal each of which may be generated by a sensor unit and/or an input unit of a control member in response to various variables (or user inputs), while a “dynamic feature” refers to a temporal feature as well as a spatial feature of movement of a single part or multiple parts of a body member.

Unless otherwise defined in the following specification, all technical and scientific terms used herein have the same meaning as commonly understood by one of ordinary skill in the art to which the present invention belongs. Although the methods or materials equivalent or similar to those described herein can be used in the practice or in the testing of the present invention, the suitable methods and materials are described below. All publications, patent applications, patents, and/or other references mentioned herein (particularly those enumerated in the above Background section) are incorporated by reference in their entirety. In case of any conflict, the present specification, including definitions, will control. In addition, the materials, methods, and examples are illustrative only and not intended to be limiting.

Other features and advantages of the present invention will be apparent from the following detailed description, and from the claims.

### BRIEF DESCRIPTION OF THE DRAWING

FIGS. 1A to 1D are partially cutaway schematic views of exemplary pelvic exercise systems including a single or multiple sensor units with variable resistances according to the present invention;

FIGS. 2A to 2D are partially cutaway schematic views of exemplary pelvic exercise systems including various sensor units disposed in different parts thereof according to the present invention;

FIGS. 3A and 3B describe partially cutaway schematic views of an exemplary pelvic exercise system including an inflatable sensor unit according to the present invention;

FIGS. 3C to 3H are partially cutaway schematic views of exemplary pelvic exercise systems including inflatable body members according to the present invention;

FIGS. 4A to 4D are partially cutaway schematic views of exemplary pelvic exercise systems including various load units disposed in various arrangements according to the present invention; and

FIG. 5 is a schematic diagram of various functional members and units of an exemplary pelvic exercise system according to the present invention.

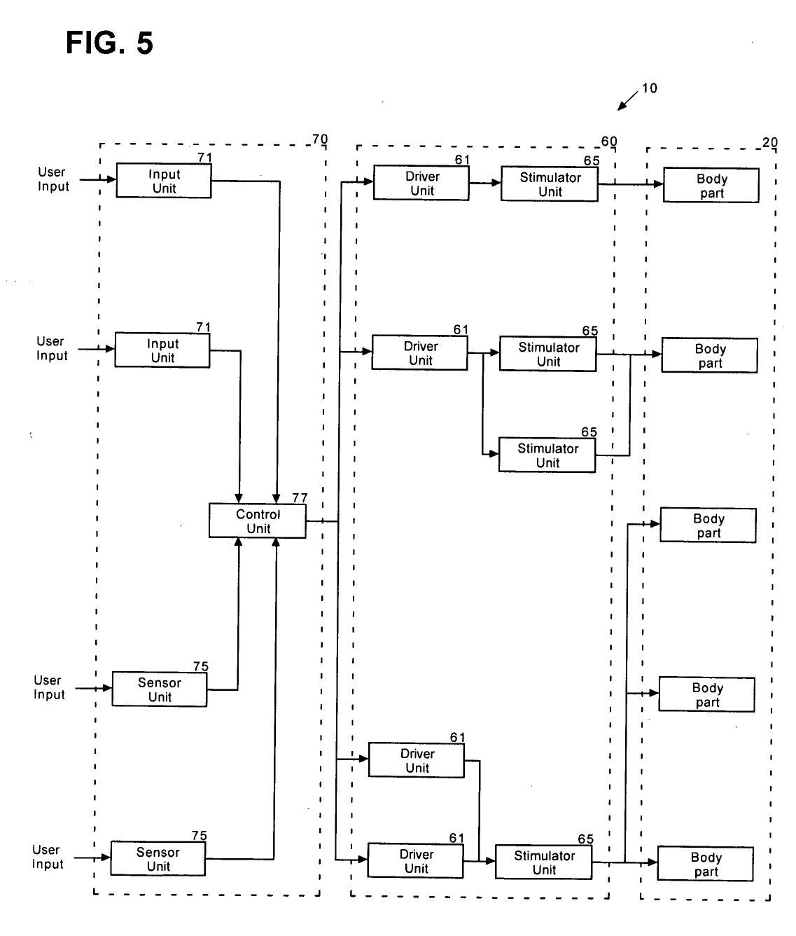

### DETAILED DESCRIPTION OF THE PREFERRED EMBODIMENTS

The present invention generally relates to a pelvic exercise system for measuring at least one variable which represents physiologic state of a pelvic structure of an user and for providing a value of the variable to the user. More particularly, the present invention relates to a pelvic exercise system for measuring the pelvic variable during exercising various muscles of the pelvic structure by at least one sensor unit of the system and for providing at least one audio and/or visual feedback signal to the user, thereby allowing the user to monitor the physiologic state of her structure. Such a system may employ various sensor units each of which may be capable of monitoring force, velocity, acceleration, displacement, duration, frequency, and electric voltages and/or current related to the pelvic muscles, where the variables may be effected by the pelvic structure or where such variables may represent reaction or resistance to various movements of the system. The system may also generate various feedback audible and/or visual signals so that the user may audibly monitor the value of the variables or that the user may perform exercise while synchronizing contraction and relaxation of the structure with such feedback signals. Such a system may be arranged to monitor the variable while the user maintains a preset posture of the pelvic structure or to measure variations in such a variable while the user changes the posture of the structure during exercise.

The present invention relates to various methods of measuring various variables representing the state of the pelvic structure and of providing the user with values of such pelvic variables. More particularly, the present invention relates to various methods for measuring various pelvic variables, various methods for acquiring values of the variables in a single portion and/or multiple portions of the structure, for generating audible and/or visual feedback signals during exercising the pelvic structure, for synchronizing such exercising with the audible and/or visual signals representing such variables, for measuring multiple variables in a single portion or multiple portions of the structure using a single or multiple sensor units, for fitting the sensor unit onto the structure during measuring the variables, and the like. The present invention also relates to various methods of improving strengths and/or tones of the pelvic muscles, of assessing such variables in a preset posture of the structure, of assessing a change in such variables as the user changes the posture, of identifying the posture and/or a series of such postures in which one or more pelvic variables may define desirable values, and the like. The present invention also relates to various processes for making various members, units, and/or parts of such pelvic exercise systems, for measuring various variables by such sensor units, for providing the values of such variables in various modes, and the like.

Various aspects and/or embodiments of various systems, methods, and/or processes of this invention will now be described more particularly with reference to the accompanying drawings and text, where such aspects and/or embodiments thereof only represent different forms. Such systems, methods, and/or processes of this invention, however, may also be embodied in many other different forms and, accordingly, should not be limited to such aspects and/or embodiments which are set forth herein. Rather, various exemplary aspects and/or embodiments described herein are provided so that this disclosure will be thorough and complete, and fully convey the scope of the present invention to one of ordinary skill in the relevant art.

Unless otherwise specified, it is to be understood that various members, units, elements, and parts of various systems of the present invention are not typically drawn to scales and/or proportions for ease of illustration. It is also to be understood that such members, units, elements, and/or parts of various systems of this invention designated by the same numerals may typically represent the same, similar, and/or functionally equivalent members, units, elements, and/or parts thereof, respectively.

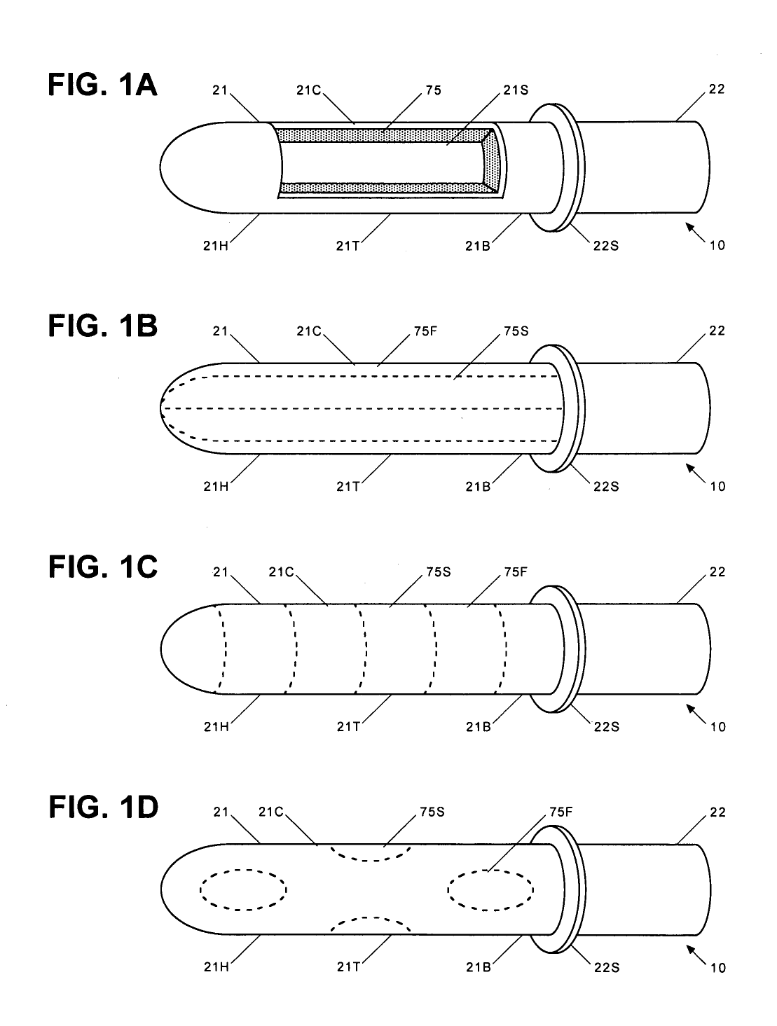

In one aspect of the present invention, pelvic exercise systems may be provided with various sensing mechanisms for measuring and monitoring physiologic states of a pelvic structure of an user in terms of variable electrical resistances of their sensor units and for providing the states to the user. FIGS. 1A to 1D show partially cutaway schematic views of exemplary pelvic exercise systems which include a single sensor unit or multiple sensor units capable of changing electrical resistance thereof in response to physiologic states of a pelvic structure of an user according to the present invention.

In one exemplary embodiment of such an aspect of the invention and as described in FIG. 1A, an exemplary pelvic relaxing system 10 includes a body member 20 with a first unit 21 and a second unit 22 as well as a control member with a sensor unit 75, where such a system 10 may also include other members and/or units as disclosed in the co-pending applications. The first unit 21 is generally elongated and arranged to be inserted into the pelvic cavity during exercise, while the second unit 22 is distally coupled to the first unit 21 and arranged to form a grip for an user during exercise. The first unit 21 is divided into a head 21H, a trunk 21T, and a base 21B. The head 21H is formed in a proximal end of the body member 20 and rounded at its tip to facilitate insertion of the body member 20 into the pelvic cavity, while the trunk 21T is connected distally to the head 21H and terminates in the base 21B which may couple with the second unit 22. The head 21H, trunk 21T, and base 21B may generally be fixedly or movably coupled to one another and form an unitary body member 20. In the alternative, the head 21H, trunk 21T, and base 21B may instead be arranged to form an unitary first unit 21. As far as the user may monitor a physiologic state of her pelvic structure during exercise, detailed construction of the body member 20 and coupling modes of their parts 21H, 21T, 21B may not be generally material to the scope of the present invention. The second unit 22 is also elongated and has a shape and size to form the grip for the user, whether the user grabs the second unit 22 with his or her thumb pointing proximally, distally, and/or laterally. The second unit 22 may movably or fixedly couple with each other and form an unitary body member 20. In the alternative, the first and second units 21, 22 may instead define an unitary body member 20. A stop 22S is also defined between the first and second units 21, 22 and extends beyond exteriors of such units 21, 22. The stop 22S is generally shaped and sized to prevent the first unit 21 from being inserted into the internal cavity of such a pelvic structure beyond a desirable distance and/or depth. It is appreciated, however, that the stop 22S may be incorporated in any location along the first and/or second units 21, 22 depending on configurations of those units 21, 22 and a maximum desired penetration depth of the first unit 21 into the internal cavity of the structure. As far as the user may monitor such physiologic states of the pelvic structure, detailed construction of and/or coupling modes between such first and second units 21, 22 are not material to the scope of the present invention. As will be described below, the pelvic exercise system 10 also includes other members and units for normal operation. When the first and/or second units 21, 22 may form cavities therein, such members and/or units may then be incorporated into one or both of such units 21, 22. In the alternative, at least one of such members and/or units may be exposed through surfaces of such units 21, 22.

The first unit 21 also includes at least one cover 21C and at least one support 21S, where the cover 21C encloses the rest of the first unit 21 therein, and where the support 21S forms a backbone of the first unit 21. The cover 21C may be made of and/or include at least one inert material such that insertion of the first unit 21 into the pelvic structure may not cause any undesirable reactions by such pelvic walls of the structure. The cover 21C may also be made to be flexible or rigid, depending upon whether various physiological variables may have be transmitted therethrough toward the sensor unit 75 when such a unit 75 may be disposed thereunder. The cover 21C may be shaped and/or sized to enclose an entire part of the first unit 21 or, in the alternative, to enclose only a selected part thereof. In addition, the cover 21C may be arranged to enclose different parts of the first unit 21 by an uniform thickness or varying thicknesses. Depending upon configuration of the first unit 21, such a cover 21C may be arranged to be symmetric with respect to a point and/or line of the first unit 21 or, alternatively, to be asymmetric. As long as the cover 21C may physically isolate an interior of the first unit 21 from the pelvic structure when engaged therewith, detailed shapes and/or sizes of the cover 21C may not be material to the scope of the present invention.

The support 21S is typically disposed inside the first unit 21 in order to provide the first unit 21 with a desired configuration. Depending on design considerations, such a support 21H may be made of and/or include at least one rigid or flexible material. An exact dimension of the support 21 typically depends upon many factors such as, e.g., a dimension of the first unit 21, desired flexibility or rigidity of the first unit 21, and the like. Such a support 21S may also assist the sensor unit 75 to monitor the pelvic variables within a preset accuracy. To this end, the support 21S may be arranged to define a preset rigidity as will be described in greater detail below.

At least one sensor unit 75 may then be incorporated between the cover 21C and support 21H so that various physiological variables of the pelvic structure may be transmitted to the sensor unit 75 through the rigid cover 21C. In the embodiment shown in FIG. 1A, the cover 21C may enclose at least a substantial part of the first unit 21 and the support 21H may extend across at least a substantial part of the first unit 21 while defining an annular gap therebetween. The sensor unit 75 may be arranged to fill the gap and to receive the physiological variables of the pelvic structure through the cover 21C disposed thereover and therearound. The sensor unit 75 is also arranged to change its configuration or to deform at least a part thereof while varying its electrical resistance in response to force applied thereto, where examples of such sensor units 75 may include, but not be limited to, conductive foams with a preset porosity, conductive elastic polymers with a preset porosity or those without including any pores, and so on. It is appreciated that the system 10 of this embodiment has a single sensor unit 75 which is disposed along the substantial part of the first unit 21.

Although not shown in the figure, the system 10 may include other members and/or unit which may output to the user values of various physiologic variables of the pelvic structure monitored by the sensor unit 75. For example, the system 10 may include at least one audio and/or visual output unit so that the values of the monitored pelvic variables may be provided to the user audibly as sounds and/or visually as images. More details of such output units will be described in greater detail below. Other configurational details of such a system 10 of FIG. 1A may be similar or identical to various systems of the co-pending applications.

In operation, the first and second units 21, 22 are fixedly or movably coupled to each other to form the body member 20. The stop 22S may be disposed between the first and second units 21, 22. The system 10 is then connected to an electric power outlet with a power supply cable (not included in the figure) or provided with a battery disposed inside the first or second unit 21, 22. The user may insert the first unit 21 of the body member 20 into a desirable depth into the internal cavity of the pelvic structure and start exercise such as, e.g., contracting and relaxing the pelvic muscles while keeping a preset posture of the pelvic structure, changing the posture of the pelvic structure by moving her legs or thighs or bending her back, and so on. When the first unit 21 is inserted into the internal cavity, the conductive foam of the sensor unit 75 begins to deform in response to a normal force applied thereto by the pelvic muscles and/or wall. Such a change in configuration of the sensor unit 75 changes the electrical resistance thereof. The control member may measure the change in the electrical resistance and assess the physiologic states of the pelvic structure from various dynamic patterns of the force applied to the sensor unit 75 through the pelvic structure. Depending upon a system configuration, the control member may output various audible and/or visual signals such that the user may monitor such physiological states of her pelvic structure. It is appreciated that such a system 10 of this embodiment includes only a single sensor unit 75 and, therefore, that the change in the electrical resistance of the sensor unit 75 may be regarded as a physiologic value representing the force which is averaged over an area in which the sensor unit 75 is disposed, across which the force is applied, and so on. Other operational details of such a system 10 of FIG. 1A may be similar or identical to various systems of the co-pending applications.

In another exemplary embodiment of this aspect of the invention and as described in FIG. 1B, an exemplary pelvic exercise system 10 includes a body member 20 with a first unit 21 and a second unit 22 as well as a control member with multiple sensor units 75. The first unit 21 is similar to that of FIG. 1A, and similarly forms a head 21H, a trunk 21T, and a base 21B, while the second unit 22 is also elongated and forms a grip for the user. A stop 22S is similarly disposed along the first and/or second units 21, 22 for preventing the user form inadvertently inserting the first unit 21 into the internal cavity beyond a desirable insertion depth. Contrary to that of FIG. 1A, however, the control member includes multiple sensor units 75 each of which may be similar or identical to that of FIG. 1A but disposed side by side around a circumference of the first unit 21. Therefore, each sensor unit 75 may longitudinally extend along at least a substantial length of the first unit 21 but angularly expand by about 60° (when the system 10 includes six identical sensor units 75A-75F). In addition, each sensor unit 75 may also be incorporated in the gap defined between the cover 21C and support 21S (not shown in the figure). Other configurational details of such a system 10 of FIG. 1A may be similar or identical to those of FIG. 1A and/or those of various systems disclosed in the co-pending applications.

In operation, the first and second units 21, 22 are coupled to each other, and the stop 22S may be disposed between such units 21, 22. As the system 10 is connected to a suitable power, the user may insert the first unit 21 of the body member 20 into a desirable depth into the internal cavity of such a structure and start exercise similar to that of FIG. 1A. As the first unit 21 is inserted into the internal cavity, the conductive foams of each sensor unit 75 similarly deforms in response to the normal force applied thereto by the pelvic muscles and/or wall. The control member may then measure changes in electrical resistance of each sensor unit 75 and assess the physiologic states of the pelvic structure based on various dynamic patterns of the force applied to each sensor unit 75 through the structure. Depending upon a system configuration, the control member may output various audible and/or visual signals such that the user may monitor the physiological states of her pelvic structure through sounds and/or images. It is appreciated that the system 10 of this embodiment includes multiple sensor units 75 disposed side by side along the circumference of the first unit 21 and, therefore, that the user may monitor the physiologic states of corresponding circumferential portions of the pelvic structure. It is to be understood, however, that the change in the electrical resistance of each of the sensor units 75 may similarly be regarded as a physiologic value representing the force which may be averaged over each area in which each sensor unit 75 is disposed and/or across which the force is applied. Thus, the system 10 of this embodiment may provide the user a spatial distribution of the physiologic states of her pelvic structure. Other operational details of the system 10 shown in FIG. 1B may be similar or identical to those of FIG. 1A and/or those of various systems of the co-pending applications.

In another exemplary embodiment of this aspect of the invention and as described in FIG. 1C, an exemplary pelvic exercise system 10 includes a body member 20 with a first unit 21 and a second unit 22 and a control member with multiple sensor units 75 all of which may be similar to those of FIGS. 1A and 1B. A stop 22S is similarly disposed along the first and/or second units 21, 22 for preventing the user form inadvertently inserting the first unit 21 into the internal cavity beyond a desirable depth of insertion. Similar to that of FIG. 1B, the control member also includes multiple sensor units 75 each of which is similar or identical to that of FIG. 1A but disposed side by side along a longitudinal axis of the first unit 21. Therefore, each sensor unit 75 may extend radially or circumferentially along at least a substantial part of a perimeter of the first unit 21 defined perpendicular to the longitudinal axis of the first unit 21. In addition, each sensor unit 75 may also be incorporated in the gap defined between the cover 21C and support 21S (not shown in the figure). Other configurational details of such a system 10 of FIG. 1B may be similar or identical to those of FIGS. 1A and 1B and/or those of various systems disclosed in the co-pending applications.

In operation, the first and second units 21, 22 are coupled to each other, and the stop 22S may be disposed between such units 21, 22. As the system 10 is connected to a suitable power, the user may insert the first unit 21 of the body member 20 into a desirable depth into the internal cavity of such a structure and start exercise similar to that of FIG. 1A. As the first unit 21 is inserted into the internal cavity, the conductive foams of each sensor unit 75 similarly deforms in response to the normal force applied thereto by the pelvic muscles and/or wall. The control member may then measure changes in electrical resistance of each sensor unit 75 and assess the physiologic states of the pelvic structure based on various dynamic patterns of the force applied to each sensor unit 75 through the structure. Depending upon a system configuration, the control member may output various audible and/or visual signals such that the user may monitor the physiological states of her pelvic structure through sounds and/or images. It is appreciated that the system 10 of this embodiment includes multiple sensor units 75 disposed side by side along the longitudinal axis of the first unit 21 in order to allow to monitor the physiologic states of corresponding longitudinal portions of the structure. It is appreciated, however, that the change in the electrical resistance of each of the sensor units 75 may similarly be regarded as a physiologic value representing the force which may be averaged over each area in which each sensor unit 75 is disposed and/or across which the force is applied. Therefore, the system 10 of this embodiment may similarly provide the user a spatial distribution of the physiologic states of her pelvic structure. Other operational details of the system 10 described in FIG. 1C may be similar or identical to those of FIGS. 1A and 1B and/or those of various systems of the co-pending applications.

In another exemplary embodiment of this aspect of the invention and as described in FIG. 1D, an exemplary pelvic exercise system 10 includes a body member 20 with a first unit 21 and a second unit 22 as well as a control member with multiple sensor units 75. The first unit 21 is similar to that of FIG. 1A, and similarly forms a head 21H, a trunk 21T, and a base 21B, while the second unit 22 is also elongated and forms a grip for the user. A stop 22S is similarly disposed along the first and/or second units 21, 22 for preventing the user form inadvertently inserting the first unit 21 into the internal cavity beyond a desirable insertion depth. Similar to those of FIGS. 1B and 1C, the control member includes multiple sensor units 75 each of which may be similar or identical to those of FIGS. 1B and 1C but may rather be disposed in a localized manner such that such sensor units 75 may measure various pelvic variables only in preset parts of the first unit 21 while being incorporated in the gap defined between the cover 21C and support 21S (not shown in the figure). Other configurational details of the system 10 of FIG. 1D may be similar or identical to those described in FIGS. 1A to 1C and/or those of various systems disclosed in the co-pending applications.

In operation, the first and second units 21, 22 are coupled to each other, and the stop 22S may be disposed between such units 21, 22. As the system 10 is connected to a suitable power, the user may insert the first unit 21 of the body member 20 into a desirable depth into the internal cavity of such a structure and start exercise similar to that of FIG. 1A. As the first unit 21 is inserted into the internal cavity, the conductive foams of each sensor unit 75 similarly deforms in response to the normal force applied thereto by the pelvic muscles and/or wall. The control member may then measure changes in electrical resistance of each sensor unit 75 and assess the physiologic states of the pelvic structure based on various dynamic patterns of the force applied to each sensor unit 75 through the structure. Depending upon a system configuration, the control member may output various audible and/or visual signals such that the user may monitor the physiological states of her pelvic structure through sounds and/or images. It is appreciated that the system 10 of this embodiment includes multiple sensor units 75 disposed only in preset parts of the first unit 21. Accordingly, the user may monitor the physiologic states of corresponding portions of the pelvic structure, where the change in the electrical resistance of each of the sensor units 75 may be regarded as a physiologic value representing the force which may be averaged over each area in which each sensor unit 75 is disposed and/or across which the force is applied. Therefore, the system 10 of this embodiment may similarly provide the user a spatial distribution of the physiologic states of her pelvic structure. Other operational details of the system 10 of FIG. 1D may be similar or identical to those of FIGS. 1A to 1C and/or those of various systems of the co-pending applications.

Configurational and/or operational variations and/or modifications of the above embodiments of the exemplary systems and various members thereof described in FIGS. 1A through 1D also fall within the scope of this invention.

The foregoing sensor units of FIGS. 1A to 1D may be constructed in a variety of mechanisms to change their electrical resistances in response to various forces applied thereto through the pelvic structure. For example and as shown in FIGS. 1A to 1D, the sensor units may be arranged to deform in response to such forces while defining a skeleton (or a matrix) and including electrically conductive materials dispersed throughout the non-conductive skeleton in a preset arrangement. Therefore, any change in a configuration of such a skeleton results in changes in distances between the conductive materials, changes in conductive paths defined inside the skeleton, and/or changes in densities of the materials, either of which may in turn result in the changes in the electrical conductivity of the sensor unit. It is to be understood that the non-conductive skeleton may define any shapes and/or sizes and may be made of any deformable materials. Such conductive materials may also be dispersed across an entire part of the sensor unit or in only a preset part thereof uniformly or non-uniformly, where the conductive materials may be particles and/or powder made from any conductive metals or polymers. When desirable, such a skeleton may also be made of and/or include at least one conductive material so that deformation of such a skeleton may vary not only the conductive paths but also the distances between the conductive materials dispersed thereacross. In another example, such sensor units may include multiple conductive materials at least one of which is arranged to move or deform with respect to the rest the stationary materials. Therefore, movement or deformation of the sensor unit may vary the distances between the conductive materials, change the conductive paths defined between such materials, and the like, thereby varying the electrical resistance of the sensor unit.

The control member including one or more sensor units defining variable electrical resistances may be arranged to assess the physiologic states of such a pelvic structure in terms of various pelvic variables. For example, the control member may monitor changes in the electrical resistance of such a sensor unit and directly assess the states of the pelvic structure in terms of such resistances. In a related example, the control member may monitor the changes in an amplitude of an electric voltage (or current) caused by changes in the resistance when an electric current (or voltage) is fixed across a testing circuit. In another example, the control member may monitor one or more configurations of the sensor unit from the changes in such resistances and assess the states of the pelvic structure from such configurations and/or changes thereof, where examples of such configurations may include, but not be limited to, a length of at least a part of the sensor unit along a longitudinal axis of the system, a length (such as height or thickness) thereof which is normal or transverse to such a longitudinal axis, a radius or diameter thereof, a cross-sectional area thereof, a volume thereof, and the like. In another example, the control member may monitor various forces applied onto the sensor unit and assess the states of the pelvic structure from one or more dynamic patterns thereof, where a few examples of such dynamic patterns may be an amplitude of such force, a direction thereof, and the like. It is to be understood that each of the above variables may represent different physiologic states of the pelvic structure. For example, various dynamic patterns of the force applied onto such a sensor unit may be dependent upon detailed sensing mechanisms of the sensor unit, whereas configurational changes of the sensor unit may be governed by mechanical characteristics of such an unit. In addition, when the sensor unit includes multiple parts made of and/or including different materials, mechanical responses to various pelvic variables thereof may also become part-specific. To the contrary, the changes in the electrical resistances of the sensor unit may then be deemed as an overall or lumped response to the pelvic variables. Accordingly, the control member may be arranged to monitor various pelvic variables and assess the physiologic states of the pelvic structure depending upon which characteristics of the structure may be of the greatest interest.

Such sensor units may be arranged to exhibit various mechanical properties. For example, the sensor unit may exhibit preferentially elastic properties so that an extent of deformation of the sensor unit may be substantially proportional to the force applied thereto. When desirable, such a sensor unit may also be arranged to exhibit viscous or dampening properties so that the extent of deformation of the sensor unit may be substantially proportional to a temporal derivative of the deformation. In case of the deformable conductive foams of FIGS. 1A to 1D, such foams may be arranged to exhibit one or both of the elastic and viscous properties by manipulating a microstructure of pores and/or a network of such pores.

As briefly described in the above figures, the sensor units with variable electrical resistances may be disposed in various arrangements. For the control member with a single sensor unit, such an unit may be disposed in any part of the first unit of the body member, where an exact location of such an unit may be determined by numerous factors such as, e.g., a penetration depth of the first unit, an orientation of the first unit, a desired portion of the pelvic structure in which such pelvic variables may be monitored, and the like. In addition, the shape and/or size of the sensor unit may be determined by an area, a location, and/or a configuration of the portion of the pelvic structure in which the variables may be monitored. Accordingly, the single sensor unit may be shaped, sized, and incorporated into a preset part of the first unit, may extend axially, circumferentially or helically along the first unit. For the control member with multiple sensor units, such units may then be disposed axially, circumferentially, helically or in combination. In the alternative, at least one of the sensor units may be disposed over at least another of such units. Such units may also be disposed in an uniform interval or at a preset non-uniform interval.

Such a sensor unit may be disposed on an exterior of the first unit in order to directly contact a corresponding portion of the pelvic structure. In reference to FIGS. 1A to 1D, such a sensor unit may be incorporated along the cover of the first unit. Alternatively, the sensor unit may be disposed under the exterior (i.e., the cover) of the first unit, in which a corresponding part of the first unit may then be arranged to transmit various pelvic variables toward the sensor unit therethrough. Similarly, when the control member may include multiple sensor units, at least two of such sensor units may be disposed in layers. As described heretofore and hereinafter, at least two of multiple sensor units may measure different pelvic variables as well.

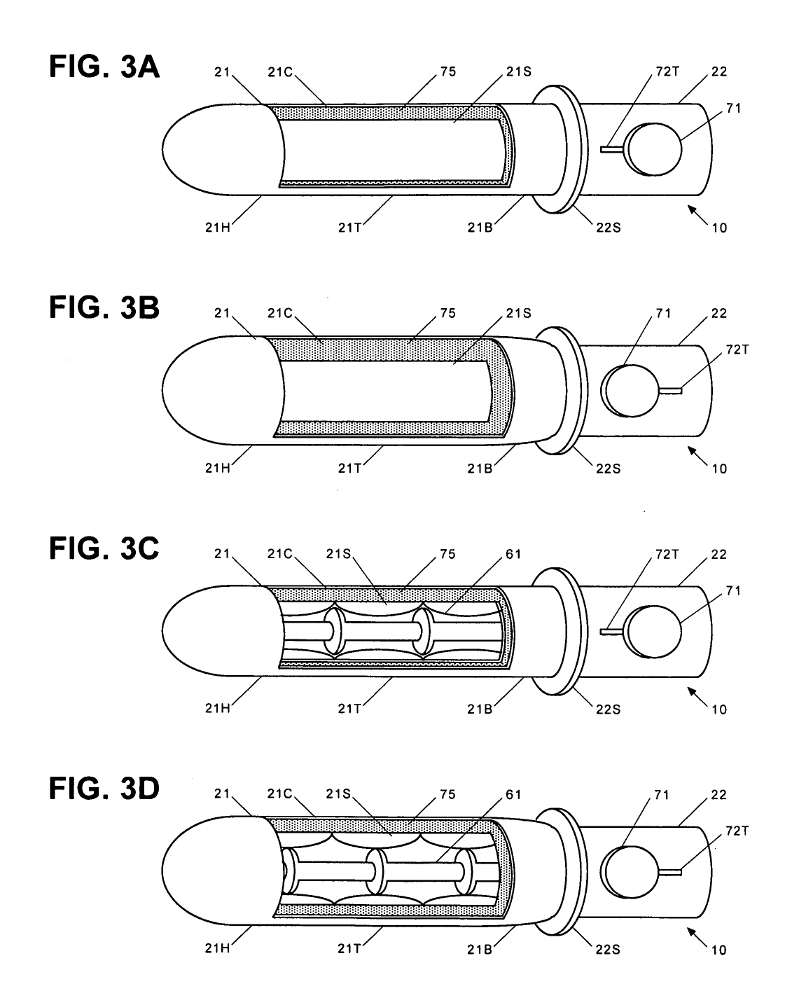

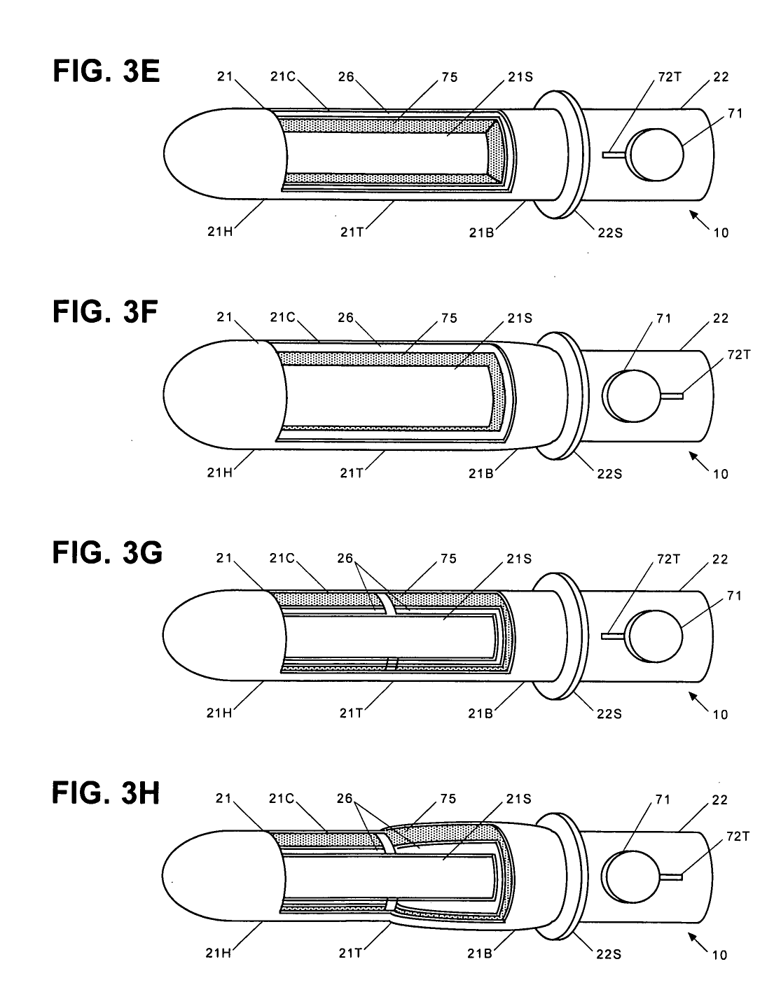

It is appreciated that, depending on users, there may exist a gap between the pelvic structure and sensor unit upon insertion of the first unit into the internal cavity of the pelvic structure. As will be described in greater detail in conjunction with FIGS. 3A to 3H, various provisions may be made in order to bring the sensor (or first) unit in contact with the pelvic structure, where such a mechanism will be referred to as “baseline adjustment” or simply as “adjustment” hereinafter throughout the description. Such adjustment may be performed by inflating or deflating at least a part of the sensor unit, inflating or deflating the cover or subjacent layers of the first unit, and the like.

Such a sensor unit may include any number of materials and/or parts which may have variable electrical resistances, where such materials and/or parts may be disposed in a preset arrangement in, over, and/or across the sensor unit. In this embodiment, each of such multiple materials and/or parts may be arranged to measure the same physiologic variable of the pelvic structure or, alternatively, to measure different pelvic variables. The control member may then operatively couple with at least two of such materials and/or parts and receive sensing signals therefrom one at a time or simultaneously. Similarly, the control member may include any number of the above sensor units each including such materials and/or parts, where such sensor units may measure the same or different pelvic variables and where the control member may receive the sensing signals therefrom simultaneously or one at a time.

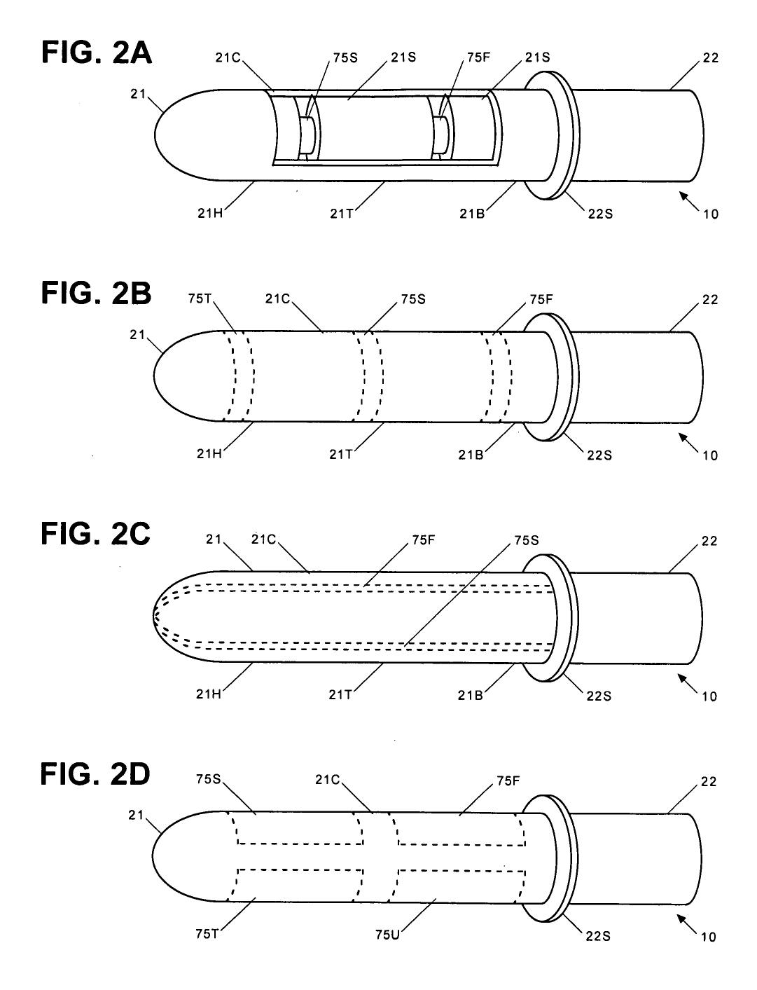

In another aspect of the present invention, pelvic exercise systems may also be provided with various sensing mechanisms for measuring and monitoring physiologic states of a pelvic structure of an user in terms of bending forces applied onto their sensor units and for providing such states to the user. FIGS. 2A to 2D show partially cutaway schematic views of exemplary pelvic exercise systems including a single sensor unit or multiple sensor units disposed in different parts thereof and capable of monitoring bending forces applied thereto according to the present invention.

In one exemplary embodiment of such an aspect of the invention and as described in FIG. 2A, an exemplary pelvic relaxing system 10 includes a body member 20 with a first unit 21 and a second unit 22 as well as a control member all of which are similar to those of FIGS. 1A to 1D. Therefore, the first unit 21 defines the head 21H, trunk 21T, and base 21B, and also couples with the second unit 22. The first unit 21 also forms the cover 21C, and the stop 22S is disposed along the first or second unit 21, 22 to prevent the first unit 21 from being inserted into the internal cavity of such a pelvic structure beyond a desirable distance and/or depth.

Inside the first unit 21 are incorporated multiple supports 21S which may similar or identical to those of FIGS. 1A to 1D and may also be rigid or flexible. Each adjacent pair of such supports 21S is coupled to each other by at least one sensor unit 75 of the control member, where such a sensor unit may be arranged to measure displacement or deformation of one of such supports 21S with respect to the other, to measure a force causing rotation or pivoting of one of such supports 21S with respect to the other, and the like. Because such sensor units 75 are arranged to measure the forces exerted onto different parts of the first unit 21, such sensor units 75 may then assess a bending force which is a result of multiple misaligned forces or which is a result of the force applied onto one end of such a first unit 21 which is fixedly supported by another end thereof. In the embodiment shown in FIG. 2A, each of three supports 21S is respectively disposed in the head 21H, trunk 21T, and base 21B of the first unit 21, and each of two sensor units 75 is disposed between the head 21H and trunk 21T of the first unit 21 and between the trunk 21T and base 21B thereof. It is appreciated that such sensor units 75 may measure such displacement or forces without resulting any deformation thereof or may do so while deforming at least a part thereof. Because such sensor units 75 may only have to measure the displacement or deformation of one support 21S with respect to the other or to monitor forces applied onto different parts of the first unit 21, such sensor units 75 may not have to be in direct contact with the pelvic structure, cover 21C of the first unit 21, and the like, as long as various pelvic variables may be transmitted to such supports 21S. In the alternative, the sensor units 75 may instead be disposed on the cover 21C of the first unit 21, inside the cover 21C or immediately below the cover 21C. When desirable, the first unit 21 may include more (or less) supports 21S and more (or less) sensor units 75 may be incorporated to measure the bending force. As described in conjunction with FIGS. 1A to 1D, including multiple sensor units 75 may allow the user to monitor a spatial distribution of such bending forces across various parts of the first unit 21. Similar to those of FIGS. 1A to 1D, such sensor units may be disposed side by side along the longitudinal axis of the first unit 21 or along the circumference thereof, thereby providing different spatial distribution of the bending forces measured along different directions. To the contrary, the control member may include a single sensor unit which may measure an overall or lumped bending force which is deemed to be averaged over a preset area or part of the first unit 21.

Although not shown in the figure, the system 10 may include other members and/or unit which may output to the user values of various physiologic variables of the pelvic structure monitored by the sensor unit 75. For example, the system 10 may include at least one audio and/or visual output unit so that the values of the monitored pelvic variables may be provided to the user audibly as sounds and/or visually as images. More details of such output units will be described in greater detail below. Other configurational details of such a system 10 of FIG. 2A may be similar or identical to various systems of the co-pending applications.

In operation, the first and second units 21, 22 are fixedly or movably coupled to each other to form the body member 20. The stop 22S may be disposed between the first and second units 21, 22. The system 10 is then connected to an electric power outlet with a power supply cable (not included in the figure) or provided with a battery disposed inside the first or second unit 21, 22. The user may insert the first unit 21 of the body member 20 into a desirable depth into the internal cavity of the pelvic structure and start exercise such as, e.g., contracting and relaxing the pelvic muscles while keeping a preset posture of the pelvic structure, changing the posture of the pelvic structure by moving her legs or thighs or bending her back, and the like. When the first unit 21 is inserted into the internal cavity of the pelvic structure, different parts of the first unit 21 receive the misaligned forces with different (or similar) amplitudes from the structure and deviate from their normal arrangement. Such displacement, deformation or movement may move or deform such supports 21S in different extents, and the sensor units 75 generate sensing signals. The control member receives and analyzes such signals and then assess various dynamic patterns of such displacement, deformation or force resulting in the bending of the first unit 21 and/or sensor units 75. Depending on a system configuration, the control member may output various audible and/or visual signals so that the user may monitor the physiological states of her pelvic structure. Other operational details of the system 10 shown in FIG. 2A may be similar or identical to those of FIGS. 1A to 1D and/or those of various systems of the co-pending applications.

In another exemplary embodiment of such an aspect of the invention and as shown in FIG. 2B, an exemplary pelvic relaxing system 10 includes a body member 20 with a first unit 21 and a second unit 22 as well as a control member all of which are similar to those of FIG. 2A. Accordingly, the first unit 21 forms the head 21H, trunk 21T, and base 21B, couples with the second unit 22, and forms the cover 21C, while the stop 22S is disposed along the first or second unit 21, 22 to prevent the first unit 21 from being inserted into the internal cavity beyond a desirable distance and/or depth.

Inside the first unit 21 and/or on its cover are incorporated multiple sensor units 75 which may be similar or identical to those of FIG. 2A and disposed between adjacent supports (not shown in this figure). Alternatively, the sensor units 75 may measure displacement thereof, deformation thereof or force applied thereto and assess therefrom the bending force causing rotation or pivoting of one part of the first unit 21 with respect to the other parts thereof. In the embodiment of FIG. 2B, each of three sensor units 75 is respectively disposed in the head 21H, trunk 21T, and base 21B of the first unit 21. It is appreciated that the sensor units 75 may measure such displacement or forces without resulting any deformation thereof or may do so while deforming at least a part thereof. Because such sensor units 75 may only have to measure the displacement or deformation of one support 21S with respect to the other or to monitor forces applied onto different parts of the first unit 21, such sensor units 75 may not have to be in direct contact with the pelvic structure, cover 21C of the first unit 21, and so on, as long as various pelvic variables may be transmitted to the supports 21S. Alternatively, the sensor units 75 may be disposed on the cover 21C of the first unit 21, in the cover 21C or immediately below the cover 21C. The first unit 21 may include a different number of sensor units 75. Depending upon a number of such sensor units 75, the control member may assess a spatial distribution of such bending forces across various parts of the first unit 21 or measure an overall or lumped bending force which is deemed to be averaged over a preset area or part of the first unit 21. Other configurational details of the system 10 of FIG. 2B are similar to those of FIGS. 1A to 1D and FIG. 2A and/or those of various systems of the co-pending applications.

In operation, the first and second units 21, 22 are fixedly or movably coupled to each other to form the body member 20. The stop 22S may be disposed between the first and second units 21, 22. The system 10 is then connected to an electric power outlet with a power supply cable (not included in the figure) or provided with a battery disposed inside the first or second unit 21, 22. The user may insert the first unit 21 of the body member 20 into a desirable depth into the internal cavity of the pelvic structure and start exercise such as, e.g., contracting and relaxing the pelvic muscles while keeping a preset posture of the pelvic structure, changing the posture of the pelvic structure by moving her legs or thighs or bending her back, and the like. When the first unit 21 is inserted into the internal cavity of the pelvic structure, different parts of the first unit 21 receive the misaligned forces with different (or similar) amplitudes from the structure, and the sensor units 75 generate sensing signals. The control member then receives and analyzes such signals and then assess various dynamic patterns of such displacement, deformation or force resulting in the bending of the first unit 21 and/or sensor units 75. Other operational details of such a system 10 shown in FIG. 2B may be similar or identical to those of FIGS. 1A to 1D and FIG. 2A and/or those of various systems of the co-pending applications.

In other exemplary embodiments of such an aspect of the invention and as shown in FIGS. 2C and 2D, each exemplary pelvic relaxing system 10 similarly includes a body member 20 with a first unit 21, a second unit 22, and a control member which are similar to those of FIGS. 2A and 2B. Therefore, the first unit 21 includes the head 21H, trunk 21T, and base 21B, couples with the second unit 22, and forms the cover 21C, and the stop 22S is disposed along the first or second unit 21, 22 to prevent the first unit 21 from being inserted into the internal cavity beyond a desirable distance or depth.

In the embodiment of FIG. 2C, multiple sensor units 75 extend axially and disposed side by side along a circumference of the first unit 21 (or angularly), where such sensor units 75 may be disposed at an uniform angle around a longitudinal axis of the first unit 21 or about non-uniform angles. Each of the sensor units 75 may extend at least a substantial length along the first unit 21 and may monitor the bending force therealong. In this context, the control member of this embodiment may assess a spatial distribution of the bending forces which are exerted by corresponding portions of the pelvic structure. In another embodiment of FIG. 2D, multiple sensor units 75 may be disposed both axially and angularly along and about the longitudinal axis of the first unit 21, respectively. Accordingly, the control member of this embodiment may assess a spatial distribution of the bending forces both along and about such a longitudinal axis of the first unit 21. Other configurational and/or operational details of such systems 10 of FIGS. 2C and 2D are also similar to those of FIGS. 1A to 1D and FIGS. 2A and 2B and/or those of various systems of the co-pending applications.

Configurational and/or operational variations and/or modifications of the above embodiments of the exemplary systems and various members thereof described in FIGS. 2A through 2D also fall within the scope of this invention.

As described herein, such sensor units may measure an extent of bending of at least a part of the first unit directly by measuring the displacement and/or deformation of such a part of the first unit. Accordingly, such a first unit may be preferably arranged to move or bend in response to the bending force applied thereto by the pelvic structure. In the alternative, the sensor units may also measure the extent of such bending indirectly by measuring the misaligned forces applied to different parts of the first (or sensor) unit and by assessing the displacement and/or deformation if the first (or sensor) unit were to be made of and/or include flexible materials. In another alternative, the sensor units may also be arranged to measure such bending force and the control member may use such bending force as the variable representing the bending capability of the pelvic structure. Therefore, the control member may monitor various pelvic variables related to such bending and assess the physiologic states of the pelvic structure depending on which bending characteristics of such a pelvic structure may be of the greatest interest.

Such sensor units may be arranged to measure various pelvic variables. For example, such a sensor unit may measure changes in lengths of the first and/or sensor units caused by displacement or deformation of such units, changes in surface areas or cross-sectional areas of the units caused thereby, changes in volumes of such units caused thereby, changes in an angle defined between at least two preset parts of such units, changes in curvature along at least a part of such units, and the like. It is appreciated that any pelvic variables defined in any directions may be measured as long as the sensor units may be properly aligned to measure such variables related to bending of such a part of the first or sensor unit. Alternatively, the control member may be arranged to assess such pelvic variables from different variables measured by such sensor units. Further details of this embodiment will be provided below.

In general, such bending is initiated by the pelvic structure and the sensor units measure such pelvic variables associated with such bending. In the alternative, the pelvic exercise system may be arranged to initiate the bending (e.g., by bending at least a part thereof) and to measure such pelvic variables which are exerted in response to such bending. In either embodiment, such variables may define various dynamic patterns as described below.

Configurational and/or operational variations and/or modifications of the above embodiments of the exemplary systems and various members thereof described in FIGS. 1A to 1D and FIGS. 2A to 2D also fall within the scope of this invention.

In addition to various sensor units disclosed in FIGS. 1A to 2D, other conventional sensors may also be incorporated into the sensor units and measure other pelvic variables, where examples of the conventional sensors may include, but not be limited to, force sensors (or force transducers), velocity sensors (or velocimeters), acceleration sensors (or accelerometers), displacement sensors capable of measuring the above variables associated with displacement or deformation of at least a part of the first and/or sensor units, contact sensors employing mechanical, electrical, magnetic, and/or chemical mechanisms, duration or timing sensors such as clocks or timers, electric current or voltage sensors (or meters) for measuring physiologic current or voltage associated with contraction and relaxation of the pelvic muscles, and the like. Any of these sensor units may then be disposed in preset locations of the first unit and in preset arrangements in order to measure various pelvic variables defined along a preset direction.

The sensor units may measure the pelvic variables in absolute values, and the control member may provide such values to the user for monitoring purposes. In the alternative, the sensor units may measure such variables in absolute values, and the control member may then convert the values into relative values by normalizing the absolute values with respect to various preset reference values of the same or different variable, where examples of such preset reference values may include, but not be limited to, a preset value of the same (or different) variable determined by a manufacturer or user, a preset value of the same (or different) variable averaged over a preset time interval and/or a preset area, a peak or maximum (or minimum) value of the same (or different) variable, a value of the same (or different) variable obtained in a proceeding measurement, and so on. Such values may further be obtained in an analog format or a digital format. In another alternative, the sensor units may measure the variables in one or more of the above relative values, where the control member may provide such relative values to the user. In addition and as described hereinabove, the sensor units and/or control member may measure and provide such absolute and/or relative values of the pelvic variables to the user without performing the baseline adjustment or after adjusting such a baseline.

The control member may include any number of such sensor units which may be disposed in almost any arrangements and which may measure the same or different variables which are defined in the same or different portions of the pelvic structure. In one example, the control member may have at least two sensor units which may monitor the same variable and may be disposed in different parts of the first unit. In another example, the control member may include at least two sensor units which may be disposed in a preset part of the first unit and measure different variables. In another example, the control member may include at least two sensor units which may be disposed in different parts of the first unit and measure different variables. When desirable, the sensor units may be disposed one over the other or side by side. In addition, at least one of such sensor units may also be disposed in the second unit and/or stop defined along the first or second unit.

As described above, such sensor units may be defined in the head, trunk, and/or base of the first unit. When the system includes multiple sensor units, at least two of such units may be identical, similar or different, at least two of such units may be disposed close to each other, away from each other, or one above the other.

The system may play sound and/or display an image of the variable and/or value thereof with an internal audio output unit, an internal visual output unit, and/or an internal audiovisual output unit. In case of storing the signals, the system may also include an internal audio input unit, an internal visual input unit, and/or an internal audiovisual input unit. Such sound and/or image may then be generated in response to the measured or assessed value of the variable which may be effected or initiated by the user or may be generated to effect such a variable with such a value by the user.

Such sensor units may be disposed in preset strategic locations along the first or second unit in order to measure the variables defined in, on or around a clitoris of the entry of the pelvic structure, a G spot on the pelvic wall, other portions of the wall, and the like.

Each of such pelvic variables may also define dynamic patterns which may be one or both of temporal patterns and spatial patterns, where examples of the temporal pattern may include, but not be limited to, an instantaneous value of the variable, a time-varying (or time-dependent) value thereof, a time-averaged value thereof, an average thereof weighted by a preset weighting functions, a peak value thereof, a time derivative thereof in the first, second or higher order, an integration thereof over time, and the like, while examples of the spatial pattern may include, but not be limited to, a localized value of the variable, a spatial distribution thereof, an area-averaged value thereof, its global or local peak in a preset domain (e.g., a preset area or volume), a spatial derivative thereof in the first, second or higher order, a spatial derivative thereof along one or more directions, an integration over a preset length, area or volume, and the like.

The temporal patterns of such pelvic variables may also include a duration of such a variable, its frequency, its temporal sequence, and the like, and the spatial patterns may include an amplitude of the variable, its direction, and the like. The dynamic pattern may further include a frequency of such a variable, its temporal rate of change (or temporal differentiation), its displacement (or its integral over time) caused thereby, and a compound value obtained by at least one of mathematical manipulation of at least one thereof. In addition, the dynamic pattern may include a duty cycle of any of the variables such as, e.g., periods in which such variables may change their amplitudes and/or direction, areas in which such variables may be defined, and the like.

In addition, such variables may include normal force applied onto at least a part of the first unit, bending force applied to such a part, axial force pulling or pushing the part into (or out of) the internal cavity, torque applied around the part, velocity of the part, acceleration of such a part, displacement of the part, contact between the part and a corresponding portion of the pelvic structure, a dimension of the portion, contraction and relaxation of the portion, a duration of at least one of such variables, a frequency of at least one of such variables, and so on. In the alternative, such variables may include normal force applied onto at least a portion of the pelvic structure, bending force applied to the portion, axial force resisting movement of the first unit into (or out of) the cavity of the pelvic structure, torque applied around such a portion, velocity of the portion, acceleration of the portion, displacement of the portion, contact between such a portion and such a part, contraction and relaxation of such a portion, a duration of at least one of such variables, a frequency of at least one of the variables, and the like. Such force may also be a torque effected about an axis of rotation or pivoting of at least a part of the sensor unit and/or at least a portion of the structure. It is appreciated that such sensor units including the force sensors may be made of and/or include elastic and/or deformable materials so as to deform in response to such force. In the alternative, such sensor units may maintain its configuration during measuring the pelvic variables by employing rigid sensors such as, e.g., piezoelectric sensors.

Each of such temporal and/or spatial patterns (i.e., the dynamic pattern) of the variable may be directly measured by the sensor unit or, alternatively, may instead be assessed from other measured patterns, where such assessment may then be performed directly by the sensor unit or by the control member. For example, the sensor unit may be the force transducer (or pressure sensor) capable of measuring various forces or pressures applied thereto or applied onto at least a portion of the pelvic structure, while the control member may assess therefrom acceleration of the sensor unit and/or the portion of the pelvic structure, velocity of the part of the sensor unit and/or of the portion of the pelvic structure, displacement of the part and/or portion, mass of the part and/or portion, momentum of such a part and/or portion, mechanical energy associated with the part and/or portion, duration of the force (or pressure), frequency of the force (or pressure), and the like. In another example, the sensor unit may be the displacement sensor capable of measuring extents of the deformation or displacement of at least a part of the sensor unit and/or at least a portion of the structure, and the control member may assess therefrom acceleration of such a part and/or portion, velocity of the part and/or portion, mass of the part and/or portion, force (or pressure) applied to the part and/or portion, momentum associated with such a part and/or portion, mechanical energy associated with the a part and/or portion, duration of the movement of such a part and/or portion effecting such displacement or deformation, frequency of such movement, and the like.

Still referring to the same variation or modification, the sensor unit may be the velocity sensor capable of measuring the velocity of at least a part of the sensor unit and/or at least a portion of the pelvic structure, while the control member may assess therefrom acceleration of such a part and/or portion, displacement of the part and/or portion, mass of the part and/or portion, force (or pressure) applied onto of the part and/or portion, momentum associated with the part and/or portion, mechanical energy associated with the part and/or portion, duration of movement of the part and/or portion which may cause such a velocity, frequency of such movement of the part and/or portion, and the like. It is appreciated that the sensor unit may measure or assess the distance to the pelvic structure, velocity of the part and/or portion, and the like, by measuring the distance to the structure. In another example, the sensor unit may also be the acceleration sensor capable of measuring the acceleration of at least a part of the sensor unit and/or at least a portion of the pelvic structure, and the control member may assess therefrom velocity of such a part and/or portion, displacement of the part and/or portion, mass of the part and/or portion, force (or pressure) applied to the part and/or portion, momentum related to the part and/or portion, mechanical energy related to the part and/or portion, duration of movement of such a part and/or portion which effects such acceleration, frequency of the movement, and the like. In another example, the sensor unit may be any conventional sensor capable of monitoring electrical, mechanical, magnetic, and/or chemical contact between at least a part of the first unit and at least a portion of the structure, while the control member may assess therefrom a duration of the contact, a frequency of such contact, and so on. When desirable, the sensor unit may include any conventional optical sensors to detect such contact. It is to be understood that such sensor units for detecting the contact may be arranged to operate based upon a preset threshold. Accordingly, the sensor unit for detecting mechanical contact may be arranged to detect such contact only when the sensor unit may be disposed within a preset distance from the pelvic structure, only when the structure exerts force defining an amplitude exceeding the threshold, and the like. Similar provisions may be applied to other sensor units for electrically, magnetically, optically, and/or chemically detecting such contact.

In addition to the above pelvic variables, the sensor units may be arranged to measure a shape and/or size of at least a portion of the pelvic structure. For example, the sensor unit may be arranged to measure the diameter or radius of a preset portion of the structure, the length or depth into a preset portion thereof, and the like. It is preferred, however, that such measurements be performed after the baseline adjustment of the sensor and/or first units so as to measure more accurate dimension of the portion of the structure.

The sensor units may also measure electric voltages and/or currents representing contraction and/or relaxation capabilities of the pelvic muscles. Any conventional voltage and/or current meters may be employed for such purposes. Alternatively, such sensor units may also measure the voltages and/or currents generated by the system and delivered to the pelvic muscles. In this embodiment, the sensor units may also measure other variables of the pelvic structure which are evoked in response to such voltages and/or currents.

Various clocks and/or timers may also be employed as the sensor units and measure various timings and/or durations associated with any of the above pelvic variables. Accordingly, such sensor units for measuring the timings, durations, and/or frequencies may be used in conjunction with other sensor units in order to determine such temporal patterns of such variables.

It is to be understood that such sensor units may measure some of the pelvic variables such as, e.g., displacement or deformation of at least a part of the sensor unit and/or at least a portion of the pelvic structure, velocity of such a part and/or portion, and acceleration of the part and/or portion, in various arrangements. In one example, the sensor units may deform or move while measuring such variables. In another example, the sensor units may instead maintain their shapes and/or sizes while measuring such variables but such sensor units may be incorporated into a preset part of the first unit which may deform or move during such measurements. In another example, both of the sensor and first units may maintain their shapes and/or sizes during such measurements.

As manifest throughout this disclosure, various sensor units of the pelvic exercise systems of this invention aim to measure various variables representing physiologic states of the pelvic structure, where such states may be elicited by, e.g., capabilities of contracting and relaxing the pelvic muscles, configurations of the pelvic structure, changes in such configurations of such a structure in different postures of the structure, changes in the configurations associated with movement of other portions of a body of the user, and the like. Accordingly, such sensor units may further be tailored in order to measure other variables representing any of the above states of the pelvic structure.

In another aspect of the present invention, pelvic exercise systems may also be provided with various mechanisms for adjusting baselines of sensor units before measuring physiologic states of a pelvic structure of an user. FIGS. 3A to 3H describe partially cutaway schematic views of exemplary pelvic exercise systems incorporating at least one mechanism for adjusting baseline according to the present invention.

In one exemplary embodiment of such an aspect of the invention and as described in FIGS. 3A and 3B, an exemplary pelvic relaxing system 10 includes a body member 20 with a first unit 21 and a second unit 22 and a control member with a sensor unit 75, where the system 10 is typically similar to that of FIG. 1A. Therefore, the first unit 21 forms the cover 21C, head 21H, trunk 21T, and base 21B, the second unit 22 forms the grip for the user, and the stop 22S is disposed along the first or second unit 21, 22. At least one sensor unit 75 may then be incorporated between the cover 21C and support 21H such that various physiological variables of the pelvic structure may be transmitted to the sensor unit 75 through the rigid cover 21C. In the embodiment shown in FIGS. 3A and 3B, the sensor unit 75 may be arranged to inflate and deflate by various mechanisms such as, e.g., filling the sensor unit 75 with air, gas, and/or fluid and discharging such therefrom. Therefore, such a sensor unit 75 may be arranged to change its shape and/or size until the sensor unit 75 may contact a preset portion of the pelvic structure. It is to be understood that the sensor unit 75 may employ any of the above sensing mechanisms. In contrary to that of FIG. 1, the second unit 22 includes at least one input unit 71 which may be arranged to move along a track 72T which is also defined along the second unit 22. Such an input unit 71 may be operatively coupled to the sensor unit 75 such that movement of the input unit 71 may change the configuration of the sensor unit 75 between its maximum and minimum configuration. Other configurational details of the system 10 of FIGS. 3A and 3B may be similar or identical to those of FIGS. 1A to 1D and FIGS. 2A to 2B, and/or those of various systems of the co-pending applications.

In operation, the first and second units 21, 22 are fixedly or movably coupled to each other to form the body member 20, and the stop 22S may be disposed between the first and second units 21, 22. After the system 10 is connected to power, the user may insert the first unit 21 into a desirable depth into the internal cavity of the pelvic structure. The user may manipulate the input unit 71 along the track 72T and increase (or decrease) a diameter of the first and/or sensor units 21, 75 until such a sensor unit 75 may snugly fit onto the pelvic wall and touch the pelvic muscles, thereby accomplishing baseline adjustment. Depending upon operating mechanisms thereof, the sensor unit may have to be adjusted so that the sensor unit having a new configuration may issue new baseline sensing signals, particularly when such a sensor unit may preferably measure the pelvic variables in absolute values. Once the sensor unit 75 may be fitted onto the pelvic structure and/or contact the pelvic structure, the user may commence pelvic exercise such as, e.g., contracting and relaxing the pelvic muscles while keeping a preset posture of the pelvic structure, varying the posture of the pelvic structure by moving her legs or thighs or bending her back, and so on. The sensor unit 75 may begin to issue the sensing signals, and the control member may measure desirable pelvic variables representing the physiologic states of the structure from various dynamic patterns thereof. Depending on a system configuration, the control member may output various audible and/or visual signals so that the user may monitor such physiological states of her structure. Further operational details of the system 10 of FIGS. 3A and 3B may also be similar or identical to those of FIGS. 1A to 1D and FIGS. 2A to 2D and/or those of various systems of the co-pending applications.

In another exemplary embodiment of such an aspect of the invention and as described in FIGS. 3C and 3D, an exemplary pelvic relaxing system 10 includes a body member 20 with a first unit 21 and a second unit 22, all of which are typically similar to that of FIG. 1A. Therefore, the first unit 21 forms the cover 21C, head 21H, trunk 21T, and base 21B, the second unit 22 forms the grip for the user, and the stop 22S is disposed along the first or second unit 21, 22. The system 10 also includes a control member with at least one sensor unit 75 which is disposed between the cover 21C and support 21S similar to that of FIGS. 3A and 3B such that various physiological variables of the pelvic structure may be transmitted to the sensor unit 75 through the rigid cover 21C. In the embodiment shown in FIGS. 3C and 3D, the sensor unit 75 may be arranged to maintain its configuration. Accordingly, the sensor unit 75 does not change its shape and/or size during measurement of various pelvic variables. Similar to that of FIGS. 3A and 3B, the sensor unit 75 of this embodiment may employ any of the above sensing mechanisms.

In contrary to that of FIGS. 3A and 3B, the support 21S may form protrusions and indentations and at least one driver unit 61 may be disposed inside and along the first unit 21. In particular, such a driver unit 61 may form protrusions and indentations matching those of the support 21S and may move or translate along a longitudinal axis of the first unit 21. Accordingly, the protrusions of the driver unit 61 may be disposed between the indentations of the support 21S when the driver unit 61 is disposed distally as exemplified in FIG. 3C, while the protrusions of the driver unit 61 may abut the indentations of the support 21S when the driver unit 61 is pushed proximally as depicted in FIG. 3D. Similar to that of FIGS. 3A and 3B, the second unit 22 includes at least one input unit 71 which may be arranged to move along a track 72T which is also defined along the second unit 22. Such an input unit 71 may be operatively coupled to the driver unit 61 such that movement of the input unit 71 may move or translate the driver unit 61 proximally or distally. It is appreciated that the sensor unit 75 of this embodiment may maintain its configuration during the movement of the driver unit 61. Further configurational details of the system 10 of FIGS. 3C and 3D may also be similar or identical to those of FIGS. 1A to 1D, FIGS. 2A to 2B, FIGS. 3A and 3B, and/or those of various systems of the co-pending applications.

In operation, the first and second units 21, 22 are fixedly or movably coupled to each other to form the body member 20, and the stop 22S may be disposed between the first and second units 21, 22. In this rest position, the protrusions of the driver unit 61 may be trapped in or near the indentations of the support 21S, thereby manipulating the first unit 21 to maintain a low profile. After the system 10 is connected to power, the user may insert the first unit 21 into a desirable depth into the cavity of the pelvic structure. The user may manipulate the input unit 71 along the track 72T and translate the driver unit 61 with respect to the support 21S so that the protrusions of the driver unit 61 may gradually abut the protrusions of the support 21S, thereby gradually increasing the profile of the first unit 21. As the sensor unit 75 may snugly fit onto the pelvic wall and touch the pelvic muscles, the baseline may then be adjusted. Depending upon operating mechanisms thereof, the sensor unit may have to be adjusted so that the sensor unit with a new configuration may issue new baseline sensing signals, particularly when such a sensor unit 75 may preferably measure the pelvic variables in absolute values. Once the sensor unit 75 may be fitted onto the pelvic structure and/or contact the pelvic structure, the user may start the pelvic exercise such as, e.g., contracting and relaxing the pelvic muscles while keeping a preset posture of the pelvic structure, varying the posture of the pelvic structure by moving her legs or thighs or bending her back, and so on. The sensor unit 75 may begin to issue the sensing signals, and the control member may measure desirable pelvic variables representing the physiologic states of the structure from various dynamic patterns thereof. Depending upon a system configuration, such a control member may output various audible and/or visual signals so that the user may monitor various physiological states of her structure. Further operational details of the system 10 of FIGS. 3C and 3D may also be similar or identical to those of FIGS. 1A to 1D, FIGS. 2A to 2D, and FIGS. 3A and 3B and/or those of various systems of the co-pending applications.

In another exemplary embodiment of such an aspect of the invention and as described in FIGS. 3E and 3F, an exemplary pelvic relaxing system 10 includes a body member 20 with a first unit 21 and a second unit 22, all of which are typically similar to that of FIG. 1A. Therefore, the first unit 21 forms the cover 21C, head 21H, trunk 21T, and base 21B, the second unit 22 forms the grip for the user, and the stop 22S is disposed along the first or second unit 21, 22. The system 10 also includes a control member with at least one sensor unit 75 which is disposed between the cover 21C and support 21S similar to that of FIGS. 3A to 3D such that various physiological variables of the pelvic structure may be transmitted to the sensor unit 75 through the rigid cover 21C. Similar to that of FIGS. 3C and 3D, the sensor unit 75 of this embodiment may also be arranged to maintain its configuration. Accordingly, the sensor unit 75 does not change its shape and/or size during measurement of various pelvic variables. Similar to that of FIGS. 3A to 3D, the sensor unit 75 of this embodiment may employ any of the above sensing mechanisms.

The support 21S of this embodiment is also similar to that of FIG. 1A and, accordingly, forms a backbone of the first unit 21. The sensor unit 75 may then be incorporated over the support 21S and below the cover 21C. However, an inflatable chamber 26 may be interposed between the sensor unit 75 and cover 21C and arranged to inflate and deflate as such a chamber 26 may be filled with and/or dispensed of air, gas, and/or fluid. Accordingly, the first unit 21 may vary its configuration until it fits onto the pelvic structure, and the sensor unit 75 may then receive various pelvic variables through the cover 21C and then through the inflatable chamber 26. Similar to those of FIGS. 3A to 3D, the second unit 22 includes at least one input unit 71 which may be arranged to move along a track 72T similarly defined along the second unit 22. The input unit 71 may operatively couple with the inflatable chamber 26 such that movement of the input unit 71 may inflate or deflate the inflatable chamber 26 and change the profile of the first unit 21. It is appreciated that the sensor unit 75 of this embodiment may maintain its configuration during the movement of the driver unit 61. Other configurational details of the system 10 of FIGS. 3E and 3F may also be similar or identical to those of FIGS. 1A to 1D, FIGS. 2A to 2B, FIGS. 3A to 3D, and/or those of various systems of the co-pending applications.

In operation, the first and second units 21, 22 are fixedly or movably coupled to each other to form the body member 20, and the stop 22S may be disposed between the first and second units 21, 22. In this rest position, the inflatable chamber 26 may be deflated in a low profile. After the system 10 is connected to power, the user may insert the first unit 21 into a desirable depth into the cavity of the pelvic structure. The user may then manipulate the input unit 71 along the track 72T and inflate (or deflate) the inflatable chamber 26 until the cover 21C or sensor unit 75 may gradually abut the pelvic structure while gradually increasing the profile of the first unit 21. When the sensor unit 75 snugly fits onto the pelvic wall and touch the pelvic muscles, the baseline may then be adjusted. Depending upon operating mechanisms thereof, the sensor unit may have to be adjusted so that the sensor unit with a new configuration may issue new baseline sensing signals, particularly when the sensor unit 75 may preferably measure the pelvic variables in absolute values. Once the sensor unit 75 is fitted onto the pelvic structure and/or contact the pelvic structure, the user may commence the pelvic exercise such as, e.g., contracting and relaxing the pelvic muscles while keeping a preset posture of the structure, varying the posture of the pelvic structure by moving her legs or thighs or bending her back, and so on. The sensor unit 75 may begin to issue the sensing signals, and the control member may measure desirable pelvic variables representing the physiologic states of the structure from various dynamic patterns thereof. Depending upon a system configuration, such a control member may output various audible and/or visual signals such that the user may monitor various physiological states of her pelvic structure. Other operational details of the system 10 of FIGS. 3E and 3F may be similar or identical to those of FIGS. 1A to 1D, FIGS. 2A to 2D, and FIGS. 3A to 3D and/or those of various systems of such co-pending applications.

In another exemplary embodiment of such an aspect of the invention and as described in FIGS. 3G and 3H, an exemplary pelvic relaxing system 10 may be generally similar to that of FIGS. 3E and 3F. However, such a first unit 21 may have a proximal inflatable chamber 26 as well as a distal inflatable chamber 26 each of which may instead be arranged to independently receive and dispense such air, gas, and/or fluid. Therefore, such a first unit 21 may be fitted onto the pelvic structure while defining different profiles in different inflatable chambers 26. Other configurational and/or operational details of the system 10 of FIGS. 3G and 3H may be similar or identical to those of FIGS. 1A to 1D, FIGS. 2A to 2D, and FIGS. 3A to 3F and/or those of various systems of such co-pending applications.

Configurational and/or operational variations and/or modifications of the above embodiments of the exemplary systems and various members thereof described in FIGS. 3A to 3H may also fall within the scope of this invention.

Regardless of such baseline adjustment, the sensor unit may be arranged to measure various pelvic variables by directly contacting a preset portion of the pelvic structure or through the inflatable chambers which may be capable of transmitting such variables to the sensor unit therethrough. Such inflatable chambers may be filled with air, gas, fluid, and/or solid capable of transmitting the variables.

Such inflation or other movements of the first unit may be generated by supplying and then by withdrawing the above transmitting media into and out of the inflatable chambers. Alternatively, such inflation or movements may be effected by the support or other parts of the first unit which may move or otherwise deform by various mechanisms.

Although not shown in these figures, the support may also be arranged to change its profile or diameter such that the sensor unit 75 disposed thereover may move toward and away from at least a portion of the pelvic structure. Such a support may be actuated either manually or by various actuator members which have been described in greater detail in the co-pending applications.

The movement or deformation of the inflatable chamber or other parts of the first unit may also be synchronized with various audio and/or visual signals provided by the system. Such movement or deformation of the first unit may also correspond to the movement of only a part of the first unit or that of an entire part of the first unit. In the alternative, the system may move not only the first unit but also at least a part of the first unit for such movement or deformation.

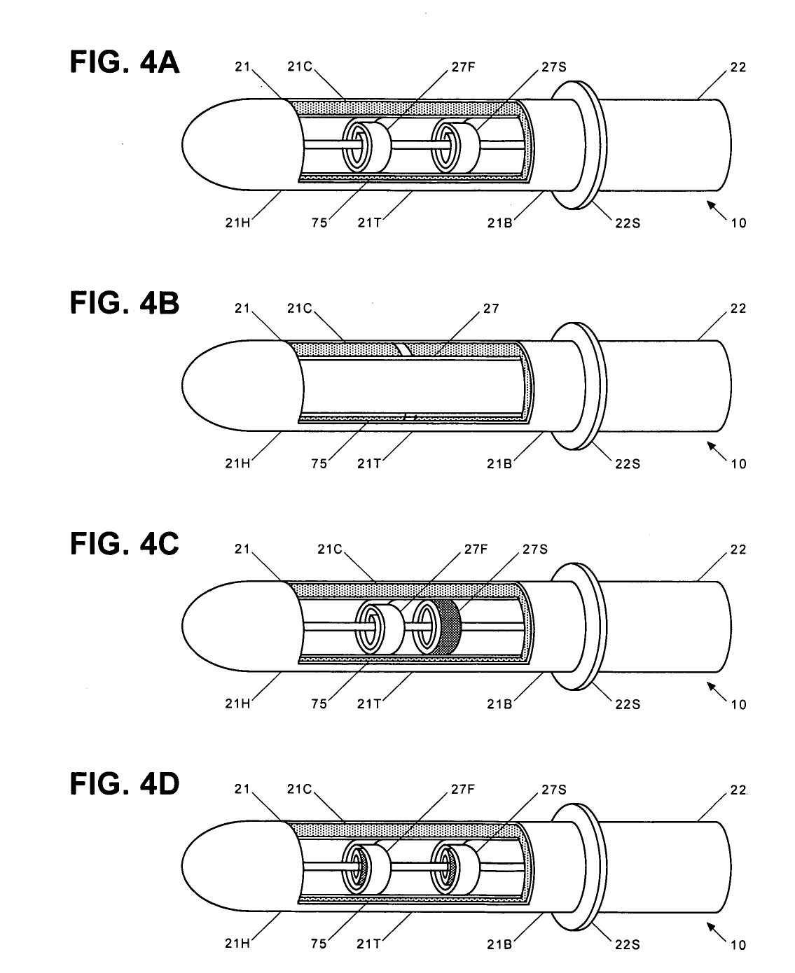

In another aspect of the present invention, pelvic exercise systems may also be provided with various load units capable of providing various elastic, viscous or viscoelastic loads to an user during exercise. FIGS. 4A to 4D are partially cutaway schematic views of various exemplary pelvic exercise systems with various load units disposed in various arrangements according to the present invention.

In one exemplary embodiment of such an aspect of the invention and as described in FIG. 4A, an exemplary pelvic relaxing system 10 includes a body member 20 with a first unit 21 and a second unit 22 and a control member with a sensor unit 75, where the system 10 is typically similar to that of FIG. 1A. Accordingly, the first unit 21 defines the cover 21C, head 21H, trunk 21T, and base 21B, the second unit 22 forms the grip for the user, and the stop 22S is disposed along the first or second unit 21, 22. At least one sensor unit 75 may also be disposed between the cover 21C and support 21H so that various physiological variables of the pelvic structure may be transmitted toward the sensor unit 75 through the rigid cover 21C.

Such a system 10 may also include multiple load units 27F, 27S which are generally disposed inside the first unit 21. In the embodiment of FIG. 4A, the load units 27F, 27S may include substantially elastic (or resistant) elements such as, e.g., coils or springs, which are disposed side by side along a longitudinal axis of the first unit 21. In addition, such elastic (or resistant) load units 27F, 27S may also operatively couple to the cover 21C and sensor unit 75 so that such units 27F, 27S may pose a preset elastic (or resistant) load to the pelvic structure during pelvic exercise. Because such load units 27F, 27S are disposed parallel to each other, the pelvic structure may be posed by an elastic (or resistant) load which may be a sum of elastic (or resistant) loads posed by each of such units 27F, 27S. Other configurational and/or operational details of the system 10 of FIG. 4A may also be similar or identical to those of FIGS. 1A to 1D, FIGS. 2A to 2B, and FIGS. 3A to 3H, and/or those of various systems of such co-pending applications.

In another exemplary embodiment of such an aspect of the invention and as shown in FIG. 4B, an exemplary pelvic relaxing system 10 may be similar to that of FIG. 4A, and include the body member 20 with the first and second units 21, 22 and the control member with the sensor unit 75. Thus, such a first unit 21 defines the cover 21C, head 21H, trunk 21T, and base 21B, the second unit 22 forms the grip for the user, and the stop 22S may be disposed along the first or second unit 21, 22. The sensor unit 75 may then be disposed between the cover 21C and support 21H such that various physiological variables of the pelvic structure may be transmitted toward the sensor unit 75 through the rigid cover 21C. Such a system 10 may also have multiple load units 27 which are generally disposed inside the first unit 21. In the embodiment of FIG. 4B, the load units 27 may be substantially elastic (or resistant) elements which are shaped as a rod or a sheet which are disposed side by side along the longitudinal axis of the first unit 21. In addition, the elastic (or resistant) load units 27 may also operatively couple to the cover 21C and sensor unit 75 so that such units 27 may pose a preset elastic (or resistant) load to the pelvic structure during pelvic exercise. Because the load units 27 are disposed parallel to each other, the pelvic structure may then be posed by an elastic (or resistant) load which may be a sum of elastic (or resistant) loads posed by each of such units 27. Further configurational and/or operational details of the system 10 of FIG. 4B may also be similar or identical to those of FIGS. 1A to 1D, FIGS. 2A to 2B, FIGS. 3A to 3H, FIG. 4A, and/or those of such systems of the co-pending applications.

In another exemplary embodiment of such an aspect of the invention and as shown in FIG. 4C, an exemplary pelvic relaxing system 10 may be similar to that of FIG. 4A, and include the body member 20 with the first and second units 21, 22 and the control member with the sensor unit 75. Thus, such a first unit 21 defines the cover 21C, head 21H, trunk 21T, and base 21B, the second unit 22 forms the grip for the user, and the stop 22S may be disposed along the first or second unit 21, 22. The sensor unit 75 may then be disposed between the cover 21C and support 21H such that various physiological variables of the pelvic structure may be transmitted toward the sensor unit 75 through the rigid cover 21C. Such a system 10 may also have multiple load units 27F, 27S which are typically disposed in the first unit 21. In the embodiment of FIG. 4C, the first load unit 27F is a substantially elastic (or resistant) element such as those of FIGS. 4A and 4B but the second load unit 27S is a substantially viscous (or dampening) element capable of dissipating at least a portion of mechanical energy applied thereto and, therefore, dampening such a portion of the mechanical energy. Such a viscous second unit 27S may employ any conventional viscous articles where air, gas, water, and/or fluid may be arranged to flow back and forth through a preset path therefor while dissipating the mechanical energy driving such a flow. Such load units 27F, 27S may also operatively couple to the cover 21C and/or sensor unit 75 so that the load units 27F, 27S may pose a preset viscoelastic load onto the pelvic structure during pelvic exercise. Because the load units 27F 27S are disposed parallel to each other, such a pelvic structure may then be posed by the viscoelastic load which may be a sum of the elastic (or resistant) load and the viscous load respectively posed by such units 27F, 27S. Other configurational and/or operational details of the system 10 of FIG. 4C may also be similar or identical to those of FIGS. 1A to 1D, FIGS. 2A to 2B, FIGS. 3A to 3H, FIGS. 4A and 4B, and/or those of such systems of the co-pending applications.

In another exemplary embodiment of such an aspect of the invention and as shown in FIG. 4D, an exemplary pelvic relaxing system 10 may be similar to that of FIG. 4C, and include the body member 20 with the first and second units 21, 22 and the control member with the sensor unit 75. Thus, such a first unit 21 defines the cover 21C, head 21H, trunk 21T, and base 21B, the second unit 22 forms the grip for the user, and the stop 22S may be disposed along the first or second unit 21, 22. The sensor unit 75 may similarly be disposed between the cover 21C and support 21H for receiving various pelvic variables through the rigid cover 21C. The system 10 may also include multiple load units 27 each of which includes a substantially elastic (or resistant) element coupling with a substantially viscous (or dampening) element in series. Such load units 27 may also operatively couple to the cover 21C and/or sensor unit 75 so that the load units 27 may pose a preset viscoelastic load onto the pelvic structure during exercise. Because such load units 27 are disposed parallel to each other, the pelvic structure may then be posed by the viscous load which may be a sum of the elastic (or resistant) loads of each load units 27. Other configurational and/or operational details of the system 10 of FIG. 4D may also be similar or identical to those described in FIGS. 1A to 1D, FIGS. 2A to 2B, FIGS. 3A to 3H, FIGS. 4A to 4C, and/or those of such systems of the co-pending applications.

Configurational and/or operational variations and/or modifications of the above embodiments of the exemplary systems and various members thereof described in FIGS. 4A to 4D may also fall within the scope of this invention.

Such a system may include any number of the above load units in any arrangements as far as the load units may pose a desirable static or dynamic load upon the exercising pelvic structure. In one example, such a system may have a constant number of viscous and/or elastic load units which may be operatively coupled to such a first unit in a fixed series, parallel or hybrid arrangement. In another example, the system may have multiple viscous and/or elastic load units at least one of which may be arranged to couple with at least another thereof in variable arrangements, e.g., by moving or changing its position, by flowing the air, gas or fluid through different paths, by varying hydraulic resistance of such paths, by modifying their coupling modes from one to the other of the series, parallel, and hybrid arrangements, and so on. Thus, an assembly of such load units may exhibit multiple viscous and/or elastic loads upon the pelvic structure. In another example, the system may be arranged to receive or discharge a certain number of such viscous and/or elastic load units so that the user may add or take away such load units at her will.

The system may also include an article of a preset mass such that the load units may exhibit a classic mass-elasticity-viscosity relationship which may be governed by a well-known second-order differential equation. Accordingly, the user may manipulate an elastic constant of the elastic load unit, a viscosity of the viscous load unit, and the mass in order to obtain desirable dynamic patterns of the assembly. As to the mass, the first unit may be tailored to serve as the article with the preset mass. In the alternative, the sensor unit may also be arranged to serve as such an article.

In another aspect of the present invention, an exemplary pelvic exercise system may have at least one body member, at least one (optional) actuator member, at least one control member, at least one power (supply) member, and the like. FIG. 2 describes a schematic diagram of various functional members and units of an exemplary pelvic exercise system according to the present invention. Such a pelvic exercise system 10 includes at least one body member 20, at least one actuator member 60, at least one control member 70, and at least one power (supply) member (not included in this figure) which may be a wire and plug assembly for receiving AC power from an electric outlet or may instead be a dry-cell battery or a rechargeable battery.

As briefly described in FIGS. 1A to 4D, the body member 20 includes at least one first unit and at least one second unit, where the first unit defines various parts capable of contacting one or more portions of the pelvic structure when engaging therewith and providing various stimuli through one or more movements thereof. The body parts with various configurations are generally similar to those of the prior art devices, whereas various novel body parts are provided in the co-pending applications.

The actuator member 60 includes at least one driver unit 61 and at least one stimulator unit 65. The main function of the actuator member 60 is to effect one or multiple movements of one or multiple parts of the body member 20 (i.e., the body parts of the first unit thereof. To this end, the driver unit 61 receives electric power from the power member and generates driving force which is transferred to the stimulator unit 65 through at least one power transmission unit (not included in this figure). The driving unit 61 may therefore be arranged to supply the air, gas, and/or fluid into the inflatable chamber and/or discharge such out of the chamber. The stimulator unit 65 may not be necessary for various pelvic exercise systems of this invention, but may be arranged to receive the driving force, optionally convert the driving force into actuating force, and then effect desirable movement of a preset part of the first unit when such a system may be used as various pelvic relaxing systems described in such co-pending applications. The stimulator unit 65 may also manipulate the driver unit 61 and change the configuration of the first unit 21. Further details for this optional actuator member have been provided in the co-pending applications as well.

The control member 70 may include at least one control unit 77 and at least one of at least one input unit 71 and sensor unit 75. The main function of the control member 70 is to generate signals for measuring various variables representing the physiological states of the pelvic structure. To this end, such a sensor unit 75 may be arranged to measure various pelvic variables and to generate sensing signals in response thereto, while the control unit 77 may be arranged to process the sensing signals, to obtain or assess the pelvic variables therefrom, and to provide the variables to the user. As have been disclosed in the first of the co-pending applications, such input and sensor units 71, 75 basically refer to similar articles capable of receiving various user inputs and then generating various signals in response thereto. Within the scope of the present invention, those articles incorporated into the first unit are to be referred to as the sensor units 75, whereas those incorporated into the second unit are to be referred to as the input units 71, unless otherwise specified. Therefore, the input unit 71 of the pelvic exercise system 10 of this invention generally stays outside the pelvic structure and receives the user input through her hand, while the sensor unit 75 of the pelvic exercise system 10 is typically disposed on, over or inside the pelvic structure and receives the user input through various portions of the pelvic structure. Other configurational details of the input unit 71 of the control member 60 have been disclosed in the co-pending applications and will be omitted for ease of illustration.

Although not shown in the figure, the control member 70 may include at least one audio and/or visual input unit, at least one audio and/or visual output unit, at least one storage unit, and at least one processing unit. The control member 70 may also include other units such as, e.g., at least one driver unit, one or more signal paths between various members and/or units of such a system 10, and so on.

The storage unit may store various audio and/or video signals temporarily or permanently. The storage unit may be any conventional data storage articles such as, e.g., magnetic tapes and/or disks, optical disks, semiconductor chips, and other data storage devices capable of storing analog and/or digital data therein. Depending on types of the devices, the control member may also include suitable drivers to operate the storage unit, where examples of such drivers may include, but not be limited to, magnetic tape or disk drivers, optical tape or disk drivers, circuits for locating and retrieving desired signals, and the like. Such signals may be classified in various modes, e.g., based upon contents of such signals, their classifications, presence or absence of action bases therein, source thereof, their voice bases, and the like. Therefore, the control member 70 may readily find and retrieve the desired signal from the storage unit. The storage unit may be disposed in various locations of the system 10 and may be exposed or hidden in the first and/or second units. When desirable, the storage unit may be provided as a replaceable cartridge so that the user may load a desired storage unit, change such a unit when used to its full capacity, and the like. The control member 70 may optionally be arranged to communicate with external storage devices in order to send, store, search, and/or retrieve desired signals thereto or therefrom.

It is appreciated within the scope of this invention that any audiovisual signal may be deemed to define at least one of a content basis, a voice basis, an action basis, and a background basis, all of which have been described in the co-pending applications. With such definitions, the processing unit receives the audiovisual signals and modifies the audio signals by changing, e.g., at least on temporal pattern thereof, their amplitudes, their frequencies, their orders, and the like. The processing unit may also generate compound signals by combining multiple audiovisual signals or, alternatively, generate a synthesized signal by changing and/or replacing at least one basis of such signals, and the like. The processing unit may receive the sensing signals from the sensor unit 75 and assess various variables therefrom as described above.

Through such audiovisual output unit and processing unit, the control member 70 may provide various audiovisual feedback signals to the user according to various modes. In one example, such a control member 70 may provide only audible signals to the user, where such signals may not include any content basis, where such signals may carry one or more content bases related to the averaged or instantaneous absolute value of the variable, the relative value of the variable compared to various preset values such as a preset reference, the user's or other's previous performances in terms of the same or different variables, and the like. The audible signal may instead carry verbal instruction about the pelvic exercise, where such instruction may be prerecorded or provided adaptively in response to the user's performance. In the alternative, such audible signals may be arranged to lead or guide the user during the pelvic practice. Such audible feedback signals are also preferably synchronized with the measured pelvic variables in order to maximize such feedback effect. In another example, such a control member 70 may provide only visual signals to the user, where such signals may be mono- or multi-chromic signals and where such signals may define a constant intensity or their intensities may vary according to various dynamic patterns of the pelvic variables. Such visual signals may similarly be arranged to provide the feedback effect to the user or, conversely, to lead or guide the user during the pelvic exercise. The visual feedback signals are also preferably synchronized with the measured pelvic variables to maximize the feedback effect. In another example, the control member 70 may also provide only tactile signals to the user, where such signals may be directed to the pelvic structure of the user, to other body parts of the user, and/or to a third party who may be assisting the user during the pelvic exercise. The tactile signals may define uniform intensities and may be applied to the same portion of the user. Alternatively, such signals may define different intensities and may be applied to the same or different portions of the user. The tactile signals may similarly be arranged to provide the feedback effect to the user or, conversely, to lead or guide the user during the pelvic exercise. Such tactile feedback signals are also preferably synchronized with the measured pelvic variables so as to maximize the feedback effect.

The control member 70 may display an image of the sensor unit 75 and then superpose such an image by the absolute and/or relative values of the measured variables, with or without displaying locations of the sensor unit 75 measuring such variables. The control member 70 may also display a static and/or dynamic image of an anatomy of the pelvic structure and identify the location of the first unit and/or sensor unit 75 in such an anatomy. This embodiment is particularly useful when the user may change her posture during the exercise and monitor changes in the measured pelvic variables in response to such changes in the postures. The user may also monitor the changes in the measured variables as she may move her leg or thigh, may bend or rotate her back, and the like.

The control member 70 and its various units may also be arranged to perform other functions so that the pelvic exercise system of this invention may also serve as various pelvic relaxing systems of the co-pending applications. In this embodiment, various members and units of the pelvic exercise system may be tailored similar to the corresponding members and units of the pelvic relaxing systems as disclosed in the co-pending applications.

The pelvic exercise system of this invention may be provided as an unitary article. In such an embodiment, the body member of the system may incorporate therein various other members and units of the system. Alternatively, the system may include the body member and the control member which may be detached from the body member and operatively couple with the body member wirelessly or through wire. When desirable, the system may be arranged to utilize external audiovisual devices for playing various sounds and/or displaying various images. Such a system may operatively couple with such external devices through wire or wirelessly.

The pelvic relaxing system of the present invention may further be construct to be waterproof. For example, various input units of the body member may be covered by a waterproof layer or may be disposed inside the second unit of the body member. In addition, the system may be arranged to run by a rechargeable battery which may be recharged by electromagnetic induction from outside.

Unless otherwise specified, various features of one embodiment of one aspect of the present invention may apply interchangeably to other embodiments of the same aspect of this invention and/or embodiments of one or more of other aspects of the present invention. Therefore, such a sensor unit of one pelvic exercise system of FIGS. 1A to 4D may be replaced interchangeably by the sensor unit of another system thereof. Similarly, various expandable mechanisms of FIGS. 3A to 3H and various load units of FIGS. 4A to 4D may be incorporated into those systems of FIGS. 1A to 2D, and the like.

The pelvic exercise system of the present invention may be used in conjunction with various audiovisual aids. For example, the user may play a source of audio signals which generates various audible sounds to which the user may synchronize her pelvic exercise, based upon which the user may perform the exercise, and the like. The control member 70 may be arranged to receive the audio signals, to compare such audio signals with the sensing signals generated by the sensor units, and to provide the user with such feedback audiovisual signals. In another example, the user may turn on a source of visual signals generating various visual signals to which such an user may synchronize the exercise, based upon which the user may perform the exercise, and the like. The control member 70 may be arranged to receive the visual signals, to compare the visual signals with the sensing signals generated by the sensor units, and then to provide the user with the feedback audiovisual signals. In yet another example, the user may play another visual signals generating various images to which the user may synchronize the pelvic exercise, based upon which the user may perform the exercise, and the like. The control member 70 may be arranged to receive the visual signals, to compare the visual signals with the sensing signals generated by the sensor units, and then to provide the user with the feedback audiovisual signals. When desirable, such images may also include conventional exercise routines such as, e.g., yoga, running, body building, and the like.

As described above, such pelvic exercise systems of this invention may be used for various purposes. For example, such pelvic exercise systems may be used to test the physiological states of various portions of the pelvic structure when the user takes a fixed posture of the pelvic structure or while the user varies the postures by movements of her various body portions. In another example, such pelvic exercise systems may assist the user to exercise thereagainst or against the viscoelastic loads posed thereby. Accordingly, the user may strengthen or tone her muscles while monitoring the changes in the physiological states of her pelvic structure. In another example, such pelvic exercise systems may further be employed to allow the user to assess an optimum posture and/or an optimum movement of a series of postures in which she may apply the optimum force onto the systems and/or may offer the optimum resistance to movements of the systems in and out of the pelvic structure. In yet another example, the pelvic exercise systems may also incorporate various conventional articles capable of providing mechanical and/or electrical stimuli to the pelvic muscles, thereby strengthening or toning such muscles to a better state.

In order to strengthen or tone the pelvic muscles and/or to assess the optimum posture and/or movement of various body parts thereof, the user may engage in various exercises while measuring the pelvic variables with the sensor units of the pelvic exercise systems of the present invention. For example, the first unit of the pelvic exercise system or an insertable part of the body member thereof may be arranged to be at least substantially stationary, i.e., such a first unit or insertable part may be arranged to not include any mobile mechanisms and, accordingly, may maintain its position during the exercise and while measuring various pelvic variables, unless such a part may be pushed out of the internal cavity by the user. It is appreciated that such a first unit or insertable part may be arranged to maintain its configuration while measuring the pelvic variables or may instead be arranged to deform or move during the measurement. The user may then vertically squeeze at least a part of the sensor and/or first units, bend or flex different parts of the sensor and/or first units, push or pull such a part along her internal cavity, rotate or pivot such a part, and the like, with or without changing the posture. In a related example, such a part of the first and/or sensor units may be shaped and/or sized so that, once inserted into the internal cavity of the pelvic structure, the first unit may tend to translate into or out of the cavity and/or may tend to rotate angularly inside the cavity. The user may then squeeze her pelvic muscles in order to retain the first unit inside her cavity, to prevent further penetration of such a first unit, to obstruct rotation of the first unit inside the cavity, and the like, with or without varying the posture. In another example, at least a part of the first unit and/or insertable part of the body member may be arranged to translate into and/or out of the internal cavity of the pelvic structure by an active mechanism. Such a system may include the actuator member which may be arranged to translate or rotate such a part into, out of or inside the internal cavity. The user may squeeze her pelvic muscles in order to retain such a first unit inside her cavity, to prevent further penetration of such a first unit, to obstruct rotation of the first unit inside the cavity, and the like, with or without varying the posture. In all of the above examples, the user may bend, rotate or otherwise move her body in order to align the first unit with a direction of gravitational force vertically, horizontally, at a preset angle, and the like.

The pelvic exercise system of this invention offers various advantages over its conventional counterparts. First of all, such a system allows the user to strengthen or tone various muscles of her pelvic structure. Secondly, such a system may provide various feedback signals to the user so as to allow the user to monitor the values of the measured or assessed variables, temporal and/or spatial patterns of a single or multiple pelvic variables, and the like. In addition, the system may also provide various feedback signals to the user as she maintains a fixed posture or changes her postures, with or without moving her legs, thighs, abdomen, and other portions of her body. Therefore, the user may assess the changes in the values of the measured or assessed pelvic variables in a certain posture or during a series of different postures. Moreover, the system may allow the user to locate her own portions through which she may sense sexual arousal or orgasms. Accordingly, the user using the pelvic exercise system of the present invention may be able to better engage in sexual activities while experiencing own pleasure and providing the maximum force to her partner in the optimum posture or a series of optimum postures.

It is appreciated that the Disclosure Documents which have been referred to in the section of “Cross-Reference” and bear the Ser. Nos. 611,016, 611,023, 611,027 and 611,331 have been referred to herein as the “co-pending applications.”

It is to be understood that, while various aspects and embodiments of the present invention have been described in conjunction with the detailed description thereof, the foregoing description is intended to illustrate and not to limit the scope of the invention, which is defined by the scope of the appended claims. Other embodiments, aspects, advantages, and modifications are within the scope of the following claims.

What is claimed is:

1. A pelvic exercise system for measuring at least one variable representing physiologic state of at least a portion of an exercising pelvic structure of an user through monitoring a change in a variable electrical resistance of at least a part thereof in response to a value of said variable and for providing said value to said user, wherein said pelvic structure defines an entry and a wall, wherein said entry is configured to form an orifice therethrough, and wherein said wall is configured to include muscles and to form an internal cavity extending inwardly and bound by said muscles, said system comprising:
at least one body member which is configured to have a first unit for contacting said portion of said pelvic structure when engaged therewith; and
wherein said sensor unit is configured to include at least one conductive foam, to define said resistance based on a dimension of said foam, and to deform said foam while varying said resistance in response to said variable of said exercising structure, and wherein said control unit is configured to operatively couple with said sensor unit and then to provide said value of said variable to said user.

2. The system of claim 1, wherein said variable is one of normal force applied to at least a part of said first unit, bending force applied to said part, axial force pulling (or pushing) said part into (or out of) said internal cavity, torque applied around said part, velocity of said part, acceleration of said part, displacement of said part, contact between said part and portion of said structure, a dimension of said portion, contraction and relaxation of said portion, a duration of at least one of said variables, and a frequency of at least one of said variables.

3. The system of claim 1; wherein said variable is one of normal force applied onto said portion of said structure, bending force applied onto said portion, axial force resisting movement of said first unit one of into and out of said cavity of said structure, torque applied around said portion, velocity of said portion, acceleration of said portion, displacement of said portion, contact between said portion and said part, contraction and relaxation of said portion, a duration of at least one of said variables, and a frequency of at least one of said variables.

4. The system of claim 2, wherein said sensor unit is configured to change said resistance as a response to said exercising pelvic structure and wherein said control unit is configured to operatively couple with said sensor unit and to provide said value of said variable in terms of said resistance to said user.

5. The system of claim 2, wherein said sensor unit is configured to effect deformation thereof while changing said resistance in response to said exercising structure and wherein said control unit is configured to operatively couple with said sensor unit and to provide said value of said variable in terms of said deformation to said user.

6. The system of claim 2, wherein said sensor unit is configured to generate force which resists said exercising structure while varying said resistance and wherein said control unit is configured to be operatively coupled with said sensor unit and to provide said value of said variable in terms of said force to said user.

7. The system of claim 2, wherein said sensor unit is configured to change said resistance as a response to at least one dynamic pattern of said exercising pelvic structure and wherein said control unit is configured to operatively couple with said sensor unit and to provide said value of said variable in terms of said dynamic pattern to said user.

8. The system of claim 2, wherein said sensor unit is configured to have a plurality of conductive foams each of which is configured to extend one of circumferentially and axially and to change said resistance based on a dimension thereof in response to said variable of said exercising structure, and wherein said control unit is configured to operatively couple with said sensor unit and to provide said value of said variable to said user.

9. The system of claim 2, wherein said sensor unit is configured to have a plurality of conductive foams at least one of each of which is configured to extend circumferentially, at least one another of which is configured to extent axially, and which are configured to vary said resistances based upon their dimensions as a response to said variable of said exercising structure, and wherein said control unit is configured to operatively couple with said sensor unit and to provide said value of said variable to said user.

10. The system of claim 2, wherein said sensor unit is configured to have a plurality of conductive foams at least one of which is configured to be disposed on an exterior of said first unit, at least one another of which is configured to be disposed under said at least one of said conductive foam, and which are configured to change said electrical resistances based upon their dimensions in response to said variable of said exercising structure, and wherein said control unit is configured to operatively couple with said sensor unit and to provide said value of said variable to said user.

11. The system of claim 2, wherein said body member is configured to incorporate therein at least one inflatable part and wherein said control unit is configured to operatively couple with said sensor unit and to provide said value of said variable to said user.

12. The system of claim 11, wherein said sensor unit is configured to have at least one part which is disposed on said inflatable part of said first unit and varying said resistance as a response to said value of said variable while said inflatable part of said first unit inflates and then deflates in response to said exercising structure.

13. The system of claim 11, wherein said sensor unit is configured to have at least one part which is disposed under an exterior of said inflatable part of said first unit and changing its resistance as a response to said value of said variable which is transmitted thereto through said inflatable part.

14. The system of claim 2, wherein said body member is configured to further include at least one load unit for resisting said exercising of said structure.

15. The system of claim 14, wherein said load unit is configured to include at least one of at least one resistant element and at least one viscous element, wherein said resistant element is configured to exhibit mechanical resistance to said exercising and wherein said viscous element is configured to induce energy dissipation during said exercising.

16. The system of claim 2, wherein said sensor unit is configured to measure a plurality of values of said variable in a plurality of portions of said structure as said user maintains a preset posture of said structure, and wherein said control unit is configured to operatively couple with said sensor unit, to assess a configuration of said structure in said posture based on said values, and to provide said user with information regarding said configuration.

17. The system of claim 2, wherein said control member is configured to have a plurality of sensor units which are configured to measure a plurality of values of said variable in a plurality of portions of said structure while said user maintains a preset posture of said structure, and wherein said control unit is configured to be operatively coupled with said sensor units, to assess a configuration of said structure in said posture based upon said values, and to provide said user with information as to said configuration.

18. The system of claim 2, wherein said control member is configured to have a plurality of sensor units which are configured to monitor a plurality of values of said variables in a plurality of portions of said structure while said user maintains a preset posture of said structure, and wherein said control unit is configured to be operatively coupled with said sensor units, to assess a configuration of said structure in said posture based upon said values, and to provide said user with information as to said configuration.

19. A pelvic exercise system for measuring at least one variable representing physiologic state of at least a portion of an exercising pelvic structure of an user, wherein said pelvic structure has an entry and a wall, wherein said entry is configured to define an orifice therethrough, and wherein said wall is configured to include muscles and to define an internal cavity extending inwardly and bound by said muscles, said system comprising:
at least one body member which is configured to have a first unit for contacting said portion of said pelvic structure when engaged therewith; and
at least one control member with at least one sensor unit and at least one control unit, wherein said sensor unit is configured to measure at least two of said variables when said user is in a preset posture of said structure, and wherein said control unit is configured to be operatively coupled to said sensor unit, to assess a configuration of said structure in said posture, and to generate and display at least one image reflecting said configuration, thereby allowing said user to monitor said configuration in said posture.

20. A method of monitoring at least one variable representing physiologic state of at least a portion of an exercising pelvic structure of an user by at least one article with variable resistance of a pelvic exercise system, wherein said structure includes an entry and a wall, wherein said entry defines an orifice therethrough, and wherein said wall includes muscles and defines an internal cavity extending inwardly from said entry and bound by said muscles, said method comprising the steps of:
forming said article from a conductive foam;
operatively coupling said article with said portion of said structure;
exercising said muscles of said structure while squeezing said article;
changing said resistance of said article in response to said squeezing; and
measuring said variable from said resistance, thereby monitoring said state of said structure during said exercising.
